(19) United States
(12) Patent Application Publication
(10) Pub. No.: US 2003/0100069 A1
(43) Pub. Date: May 29, 2003

(54) Human tumor necrosis factor receptor-like protein 8

(75) Inventors: Jian Ni, Germantown, MD (US); Paul A. Moore, Germantown, MD (US)

Correspondence Address: HUMAN GENOME SCIENCES INC, 9410 KEY WEST AVENUE, ROCKVILLE, MD, 20850

(73) Assignee: Human Genome Sciences, Inc., Rockville, MD

(21) Appl. No.: 10/291,480

(22) Filed: Nov. 12, 2002

Related U.S. Application Data
(62) Division of application No. 09/768,779, filed on Jan. 25, 2001.
Application No. 09/768,779 is a continuation of application No. 09/086,582, filed on May 29, 1998, now abandoned.
(60) Provisional application No. 60/048,020, filed on May 29, 1997.

(51) Int. Cl. A61K 38/17; C07K 14/715; C07H 21/04; C12P 21/02; C12N 5/06

(52) U.S. Cl. 435/69.1; 435/320.1; 435/325; 514/12; 530/350; 536/23.5

(57) ABSTRACT

The present invention relates to novel members of the Tumor Necrosis Factor family of receptors. The invention provides isolated nucleic acid molecules encoding human TR8 receptors. TR8 polypeptides are also provided, as are vectors, host cells and recombinant methods for producing the same. The invention further relates to screening methods for identifying agonists and antagonists of TR8 receptor activity. Also provided are diagnostic methods for detecting disease states related to the aberrant expression of TR8 receptors. Further provided are therapeutic methods for treating disease states related to aberrant proliferation and differentiation of cells which express the TR8 receptors.

### CROSS-REFERENCE TO RELATED APPLICATIONS

This application is a divisional of and claims benefit under 35 U.S.C. §120 to U.S. application Ser. No. 09/768,779, filed Jan. 25, 2001; which is a continuation of and claims benefit under 35 U.S.C §120 to U.S. application Ser. No. 09/086,582, filed May 28, 1998; which in turn claims the benefit under 35 U.S.C. §119(e) of the filing date of Provisional Application No. 60/048,020, filed May 29, 1997, each of which documents are herein incorporated by reference in their entireties.

### FIELD OF THE INVENTION

The present invention relates to novel members of the Tumor Necrosis Factor (TNF) receptor family. More specifically, isolated nucleic acid molecules are provided encoding a human TNF receptor-related protein, referred to herein as the TR8 receptor of FIGS. 1A-C, having considerable homology to human type-2 TNF receptor (TNF-R11). TR8 polypeptides are also provided. Further provided are vectors, host cells and recombinant methods for producing the same. The invention also relates to both the inhibition and enhancement of the activity of TR8 receptor polypeptides and diagnostic methods for detecting TR8 receptor gene expression.

### BACKGROUND OF THE INVENTION

Human tumor necrosis factors α (TNF-α) and β (TNF-β or lymphotoxin) are related members of a broad class of polypeptide mediators, which includes the interferons, interleukins and growth factors, collectively called cytokines (Beutler, B. and Cerami, A., Annu. Rev. Immunol., 7:625-655 (1989)).

Tumor necrosis factor (TNF-α and TNF-β) was originally discovered as a result of its anti-tumor activity, however, now it is recognized as a pleiotropic cytokine playing important roles in a host of biological processes and pathologies. To date, there are ten known members of the TNF-related cytokine family, TNF-α, TNF-β(lymphotoxin-α), LT-β, TRAIL and ligands for the Fas receptor, CD30, CD27, CD40 (also known as CDw40), OX40 and 4-1BB receptors. These proteins have conserved C-terminal sequences and variable N-terminal sequences which are often used as membrane anchors, with the exception of TNF-β. Both TNF-α and TNF-β function as homotrimers when they bind to TNF receptors.

TNF is produced by a number of cell types, including monocytes, fibroblasts, T-cells, natural killer (NK) cells and predominately by activated macrophages. TNF-α has been reported to have a role in the rapid necrosis of tumors, immunostimulation, autoimmune disease, graft rejection, producing an anti-viral response, septic shock, cerebral malaria, cytotoxicity, protection against deleterious effects of ionizing radiation produced during a course of chemotherapy, such as denaturation of enzymes, lipid peroxidation and DNA damage (Nata et al., J. Immunol. 136(7):2483 (1987)), growth regulation, vascular endothelium effects and metabolic effects. TNF-α also triggers endothelial cells to secrete various factors, including PAI-1, IL-1, GM-CSF and IL-6 to promote cell proliferation. In addition, TNF-α up-regulates various cell adhesion molecules such as E-Selectin, ICAM-1 and VCAM-1. TNF-α and the Fas ligand have also been shown to induce programmed cell death.

TNF-β has many activities, including induction of an antiviral state and tumor necrosis, activation of polymorphonuclear leukocytes, induction of class I major histocompatibility complex antigens on endothelial cells, induction of adhesion molecules on endothelium and growth hormone stimulation (Ruddle, N. and Homer, R., Prog. Allergy 40:162-182 (1988)).

Both TNF-α and TNF-β are involved in growth regulation and interact with hemopoietic cells at several stages of differentiation, inhibiting proliferation of various types of precursor cells, and inducing proliferation of immature myelomonocytic cells. Porter, A., Tibtech 9:158-162 (1991).

Recent studies with “knockout” mice have shown that mice deficient in TNF-β production show abnormal development of the peripheral lymphoid organs and morphological changes in spleen architecture (reviewed in Aggarwal et al., Eur Cytokine Netw, 7(2):93-124 (1996)). With respect to the lymphoid organs, the popliteal, inguinal, para-aortic, mesenteric, axillary and cervical lymph nodes failed to develop in TNF-β −/− mice. In addition, peripheral blood from TNF-β −/− mice contained a three-fold reduction in white blood cells as compared to normal mice. Peripheral blood from TNF-β −/− mice, however, contained four fold more B cells as compared to their normal counterparts. Further, TNF-β, in contrast to TNF-α, has been shown to induce proliferation of EBV-infected B cells. These results indicate that TNF-β is involved in lymphocyte development.

The first step in the induction of the various cellular responses mediated by TNF-α or TNF-β is their binding to specific cell surface or soluble receptors. Two distinct TNF receptors of approximately 55-KDa (TNF-R1) and 75-KDa (TNF-R11) have been identified (Hohman et al., J. Biol. Chem., 264:14927-14934 (1989)), and human and mouse cDNAs corresponding to both receptor types have been isolated and characterized (Loetscher et al., Cell, 61:351 (1990)). Both TNF-Rs share the typical structure of cell surface receptors including extracellular, transmembrane and intracellular regions.

These molecules exist not only in cell bound forms, but also in soluble forms, consisting of the cleaved extra-cellular domains of the intact receptors (Nophar et al., EMBO Journal, 9 (10):3269-76 (1990)) and otherwise intact receptors wherein the transmembrane domain is lacking. The extracellular domains of TNF-RI and TNF-RII share 28% identity and are characterized by four repeated cysteine-rich motifs with significant intersubunit sequence homology. The majority of cell types and tissues appear to express both TNF receptors and both receptors are active in signal transduction, however, they are able to mediate distinct cellular responses. Further, TNF-RII was shown to exclusively mediate human T-cell proliferation by TNF as shown in PCT WO 94/09137.

TNF-RI dependent responses include accumulation of C-FOS, IL-6, and manganese superoxide dismutase mRNA, prostaglandin E2 synthesis, IL-2 receptor and MHC class I and II cell surface antigen expression, growth inhibition, and cytotoxicity. TNF-RI also triggers second messenger systems such as phospholipase A, protein kinase C, phosphatidylcholine-specific phospholipase C and sphingomyelinase (Pfefferk et al., Cell, 73:457-467 (1993)).

Several interferons and other agents have been shown to regulate the expression of TNF receptors. Retinoic acid, for example, has been shown to induce the production of TNF receptors in some cells type while down regulating production in other cells. In addition, TNF-α has been shown to affect the localization of both types of receptor. TNF-α induces internalization of TNF-RI and secretion of TNF-RII (reviewed in Aggarwal et al., supra). Thus, the production and localization of both TNF-Rs are regulated by a variety of agents.

Both the yeast two hybrid system and co-precipitation and purification have been used to identify ligands which associate with both types of the TNF-Rs (reviewed in Aggarwal et al., supra and Vandenabeele et al, Trends in Cell Biol. 5:392-399 (1995)). Several proteins have been identified which interact with the cytoplasmic domain of a murine TNF-R. Two of these proteins appear to be related to the baculovirus inhibitor of apoptosis, suggesting a direct role for TNF-R in the regulation of programmed cell death.

### SUMMARY OF THE INVENTION

The present invention provides isolated nucleic acid molecules comprising polynucleotides encoding a TR8 receptor having the amino acid sequence shown in FIGS. 1A-C (SEQ ID NO:2) or the amino acid sequence encoded by the cDNA clone encoding the TR8 receptor deposited in a bacterial vector as ATCC Deposit Number 97956 on Mar. 13, 1997. The present invention also relates to recombinant vectors, which include the isolated nucleic acid molecules of the present invention, and to host cells containing the recombinant vectors, as well as to methods of making such vectors and host cells and for using them for production of TR8 polypeptides or peptides by recombinant techniques.

The invention further provides isolated TR8 polypeptides having amino acid sequences encoded by the polynucleotides described herein.

The present invention also provides a screening method for identifying compounds capable of enhancing or inhibiting a cellular response induced by TR8 receptors, which involves contacting cells which express TR8 receptors with the candidate compound, assaying a cellular response, and comparing the cellular response to a standard cellular response, the standard being assayed when contact is made in absence of the candidate compound; whereby, an increased cellular response over the standard indicates that the compound is an agonist and a decreased cellular response over the standard indicates that the compound is an antagonist.

In another aspect, a screening assay for agonists and antagonists is provided which involves determining the effect a candidate compound has on the binding of cellular ligands to TR8 receptors. In particular, the method involves contacting TR8 receptors with a ligand polypeptide and a candidate compound and determining whether ligand binding to the TR8 receptors is increased or decreased due to the presence of the candidate compound.

The invention further provides a diagnostic method useful during diagnosis or prognosis of a disease states resulting from aberrant cell proliferation due to alterations in TR8 receptor expression.

An additional aspect of the invention is related to a method for treating an individual in need of an increased level of a TR8 receptor activity in the body comprising administering to such an individual a composition comprising a therapeutically effective amount of isolated TR8 polypeptides of the invention or an agonist thereof.

A still further aspect of the invention is related to a method for treating an individual in need of a decreased level of a TR8 receptor activity in the body comprising, administering to such an individual a composition comprising a therapeutically effective amount of a TR8 receptor antagonist.

The invention additionally provides soluble forms of the polypeptides of the present invention. Soluble peptides are defined by amino acid sequences wherein the sequence comprises the polypeptide sequence lacking a transmembrane domain. Such soluble forms of the TR8 receptors are useful as antagonists of the membrane bound forms of the receptors.

### BRIEF DESCRIPTION OF THE FIGURES

FIGS. 1A-C shows the nucleotide sequence (SEQ ID NO:1) and deduced amino acid (SEQ ID NO:2) sequence of a TR8 receptor. Three potential secretory leader sequences have been predicted for the complete polypeptide, of about 21, 23 or 25 amino acid residues, of which the longest, from amino acid 1 to 25 in FIGS. 1A-C, is underlined (amino acid residue −25 to −1 in SEQ ID NO:2). The deduced complete amino acid sequence includes 615 amino acid residues and has a deduced molecular weight of about 65,940 Da. It is further predicted that amino acid residues from about 26 to about 211 in FIGS. 1A-C (amino acid residues 1 to 186 in SEQ ID NO:2) constitute the extracellular domain; from about 212 to about 230 (amino acid residues 187 to 205 in SEQ ID NO:2) the transmembrane domain (underlined); and from about 231 to about 615 (amino acid residues 206 to 590 in SEQ ID NO:2) the intracellular domain.

FIG. 2 shows the regions of similarity between the amino acid sequences of the TR8 receptor protein of FIGS. 1A-C (labeled HDPIK17xxb protein) and a human TNF Receptor II protein (SEQ ID NO:3) which is labeled “TNFR2” (GenBank Accession Number M55994) (percent similarity: 46.847; percent identity: 30.405).

FIGS. 3A-C shows an alignment of the amino acid sequences of the TR8 receptor protein of FIGS. 1A-C (labeled HDPIK17xxb) and a human TNF Receptor II protein (SEQ ID NO:3), a human CD40 protein (SEQ ID NO:4; GenBank Accession Number X60592), a human “OX40” surface antigen protein (SEQ ID NO:5, GenBank Accession Number S76792), and a human lymphotoxin-beta (“LTbetaR”) (SEQ ID NO:6; GenBank Accession Number L04270) (white letters in solid black boxes represent residues that match the consensus exactly).

FIG. 4 shows a structural analysis of the TR8 receptor amino acid sequence of FIGS. 1A-C. Alpha, beta, turn and coil regions; hydrophilicity and hydrophobicity; amphipathic regions; flexible regions; antigenic index and surface probability are shown. In the “Antigenic Index—Jameson-Wolf” graph, amino acid residues 35 to 90, 107 to 210, 236 to 282, 292 to 537 and 556 to 615 in FIGS. 1A-C and FIGS. 3A-C (amino acid residues 10 to 65, 82 to 185, 211 to 257, 267 to 512, and 531 to 590 in SEQ ID NO:2) correspond to the shown highly antigenic regions of the TR8 receptor protein.

FIGS. 5A-B. Schematic diagram of TR8 and its deletions variants.

FIG. 5A.—TR8 and its deletion mutants were fused with a FLAG epitope tag at the N-terminus using the signal sequence in the expression vector pCMVFLAG1 as described in Example 6 under Experimental Procedures. The recited amino acid position corresponds to that depicted in FIGS. 1A-C and FIGS. 3A-C. The Roman numerals I, II, and III represent TRAF binding domains within the cytoplasmic domain of TR8.

FIG. 5B.—Amino acid sequence alignment of the TRAF binding domains in various TNFR family members (SEQ ID NOS:18-21) and in human TR8 (SEQ ID NOS:22-24). The recited amino acid position of TR8 corresponds to that depicted in SEQ ID NO:2.

### DETAILED DESCRIPTION OF THE PREFERRED EMBODIMENTS

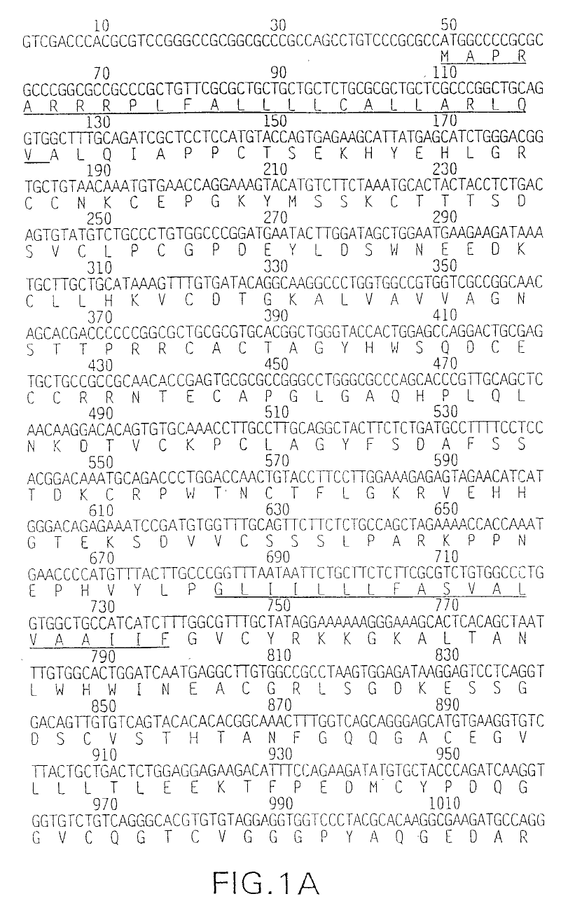

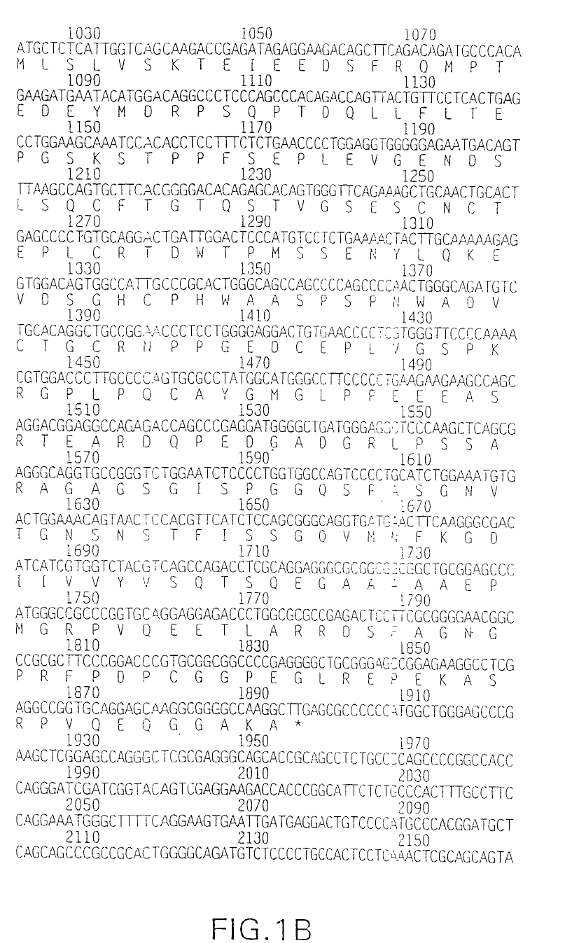

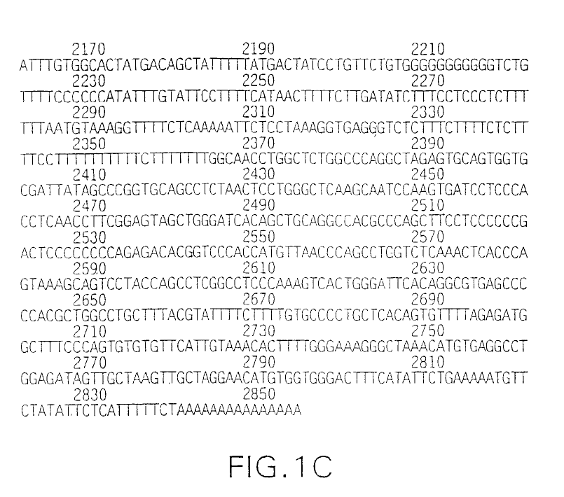

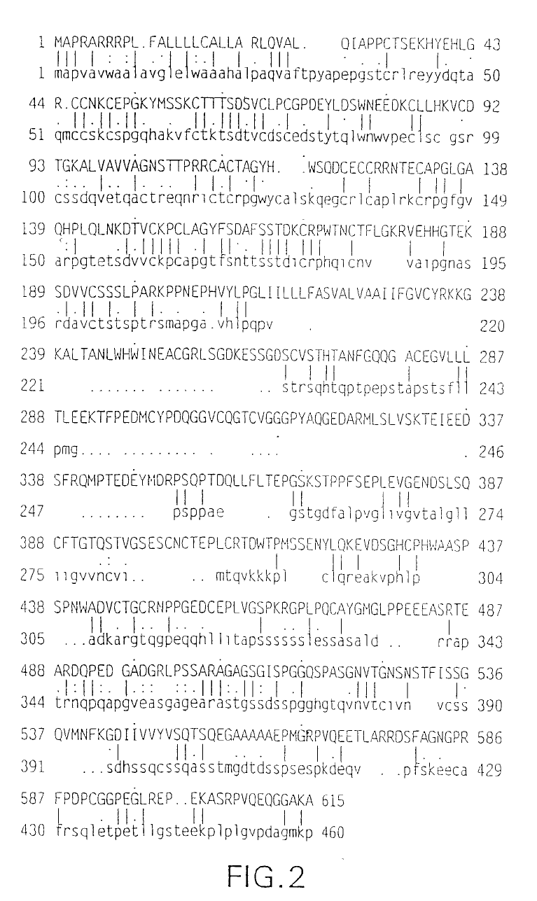

The present invention provides isolated nucleic acid molecules comprising polynucleotides encoding a TR8 polypeptide (FIGS. 1A-C (SEQ ID NO:2)), the amino acid sequence of which was determined by sequencing a cloned cDNA. The TR8 protein shown in FIGS. 1A-C shares sequence homology with the human TNF receptor II (FIG. 2 (SEQ ID NO:3)). On Mar. 13, 1997 a deposit was made at the American Type Culture Collection, 10801 University Boulevard, Manassas, Va. 20110-2209, USA, and given accession number 97956. The nucleotide sequence shown in FIGS. 1A-C (SEQ ID NO:1) was obtained by sequencing a cDNA clone (Clone ID HDPIK17) containing the same amino acid coding sequences as the clone in ATCC Accession No. 97956. The deposited clone is contained in the pBluescript SK(−) plasmid (Stratagene, La Jolla, Calif.).

As used herein, “TR8 protein”, “TR8 receptor”, receptor protein”, “TR8”, and “TR8 polypeptide” refer to all proteins resulting from the alternate splicing of the genomic DNA sequences encoding proteins having regions of amino acid sequence identity and receptor activity which correspond to the protein shown in FIGS. 1A-C (SEQ ID NO:2). The TR8 protein shown in FIGS. 1A-C is an example of such a receptor protein.

Nucleic Acid Molecules

Unless otherwise indicated, all nucleotide sequences determined by sequencing a DNA molecule herein were determined using an automated DNA sequencer (such as the Model 373 from Applied Biosystems, Inc.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

Using the information provided herein, such as the nucleotide sequence in FIGS. 1A-C, nucleic acid molecules of the present invention encoding TR8 polypeptides may be obtained using standard cloning and screening procedures, such as those used for cloning cDNAs using mRNA as starting material. Illustrative of the invention, the nucleic acid molecule described in FIGS. 1A-C (SEQ ID NO:1) was discovered in a cDNA library derived from primary dendritic cells. Other cDNA clones (two) encoding the TR8 polypeptide shown in FIGS. 1A-C were found only in the same cDNA library. One of these exhibited the following changes in the nucleotide and amino acid sequences compared to those shown in FIGS. 1A-C (SEQ ID NOS:1 and 2): an extra CGC codon was inserted after nucleotide 72 resulting in insertion of an additional R residue (after position −18 in SEQ ID NO:2); nucleotide 763 was G instead of A, resulting in the amino acid E instead of K (at position 214 in SEQ ID NO:2); and nucleotide 1583 was G instead of T, resulting in the amino acid S instead of I (at position 487 in SEQ ID NO:2).

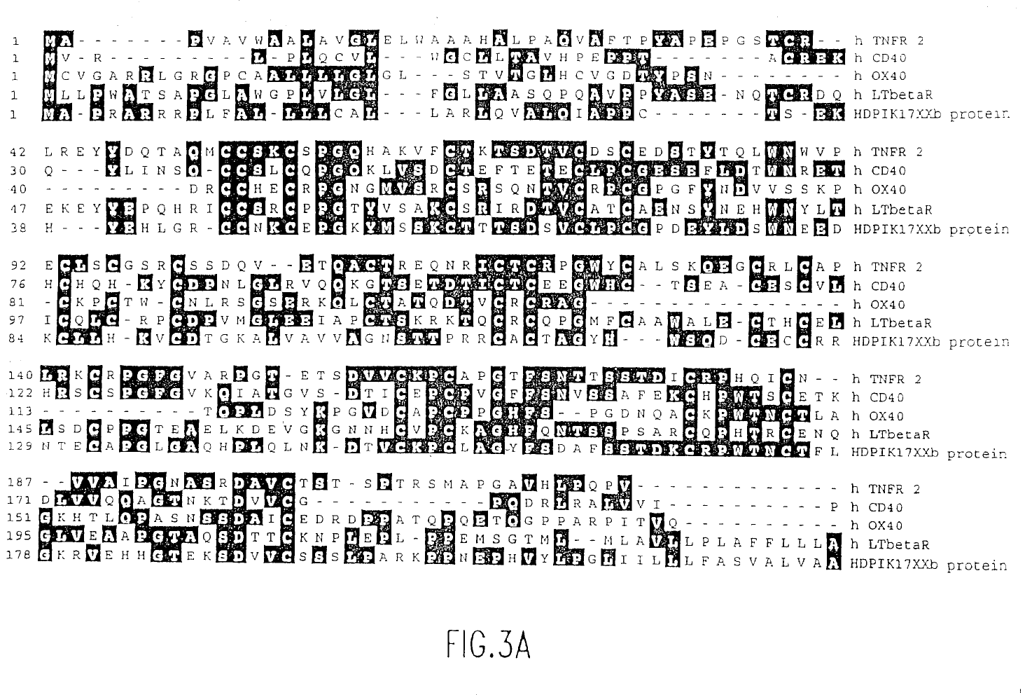

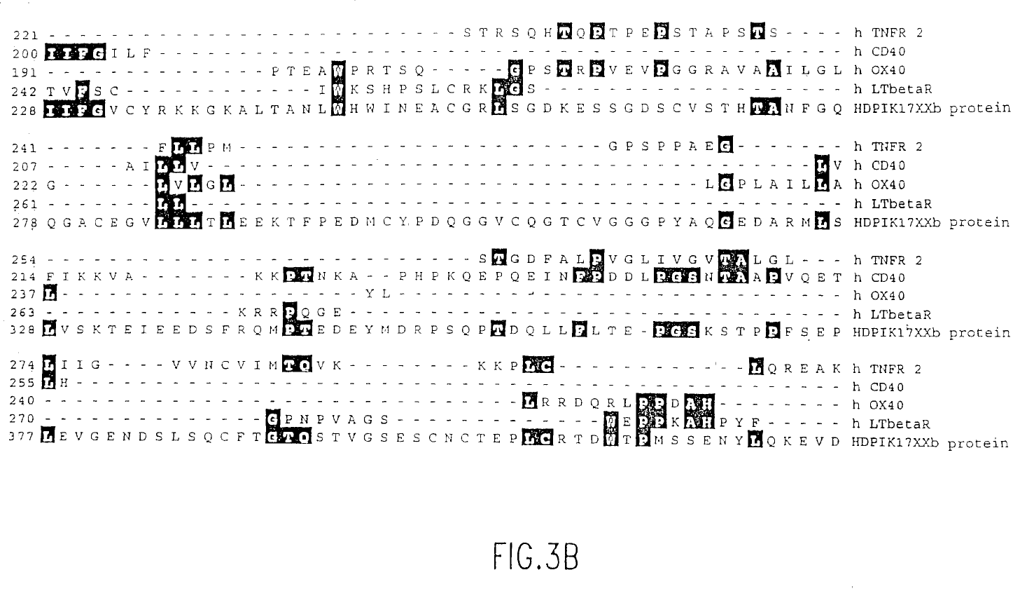

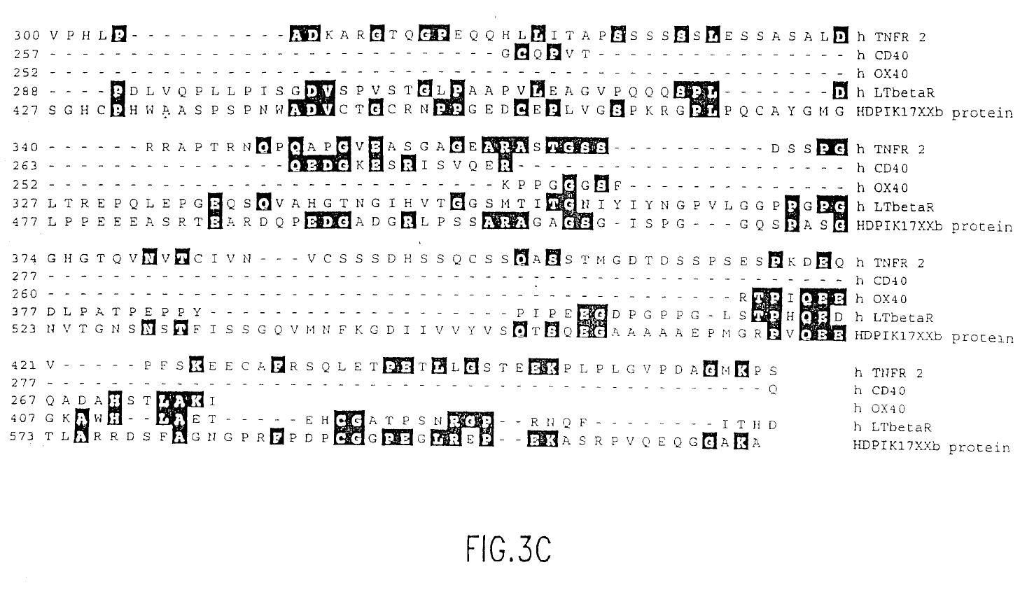

The determined nucleotide sequence of the TR8 cDNA of FIGS. 1A-C (SEQ ID NO:1) contains an open reading frame encoding a protein of about 615 amino acid residues, with three potential predicted leader sequences of about 21, 23 or 25 amino acid residues, and a deduced molecular weight of about 65,940 Da. Consistent with this deduced amino acid sequence, expression of a TR8 cDNA clone in a coupled transcription-translation system generated a protein of approximately 70 kDa, which corresponds well with the predicted molecular weight given the limits of accuracy for this determination. The amino acid sequence of the shortest potential predicted mature TR8 receptor is shown in FIGS. 1A-C, from amino acid residue about 26 to residue about 615 (amino acid residues 1 to 590 in SEQ ID NO:2). The TR8 protein shown in FIGS. 1A-C (SEQ ID NO:2) is about 30.4% identical and about 46.9% similar to the human TNF Receptor II protein shown in SEQ ID NO:3 (see FIG. 2) using the computer program “Bestfit” (see below). Using a similar alignment program with somewhat different similarity scoring rules (DNA Star “Megalign”) the TR8 protein of FIGS. 1A-C is about 71.5% similar to the same TNF Receptor II protein (SEQ ID NO:3), 61.9% similar to a human CD40 protein (SEQ ID NO:4), 71.7% similar to a human OX40 protein (SEQ ID NO:5) and 72.9% similar to a human lymphotoxin-beta receptor protein (SEQ ID NO:6); see FIGS. 3A-C.

As indicated, the present invention also provides mature forms of the TR8 receptor of the present invention. According to the signal hypothesis, proteins secreted by mammalian cells have a signal or secretory leader sequence which is cleaved from the mature protein once export of the growing protein chain across the rough endoplasmic reticulum has been initiated. Most mammalian cells and even insect cells cleave secreted proteins with the same specificity. However, in some cases, cleavage of a secreted protein is not entirely uniform, which results in two or more mature species on the protein. Further, it has long been known that the cleavage specificity of a secreted protein is ultimately determined by the primary structure of the complete protein, that is, it is inherent in the amino acid sequence of the polypeptide. Therefore, the present invention provides nucleotide sequences encoding mature TR8 polypeptides having the amino acid sequences encoded by the cDNA clone contained in the host identified as ATCC Deposit Number 97956 and as shown in FIGS. 1A-C (SEQ ID NO:2). By the mature TR8 polypeptide having the amino acid sequences encoded by the cDNA clone contained in the host identified as ATCC Deposit Number 97956 is meant the mature form(s) of the TR8 receptor produced by expression in a mammalian cell (e.g., COS cells, as described below) of the complete open reading frame encoded by the human DNA sequence of the clone contained in the vector in the deposited host.

Methods for predicting whether a protein has a secretory leader as well as the cleavage point for that leader sequence are available. For instance, the methods of McGeoch (Virus Res. 3:271-286 (1985)) and von Heinje (Nucleic Acids Res. 14:4683-4690 (1986)) can be used. The accuracy of predicting the cleavage points of known mammalian secretory proteins for each of these methods is in the range of 75-80%. von Heinje, supra. However, the two methods do not always produce the same predicted cleavage point(s) for a given protein.

In the present case, the predicted amino acid sequence of the complete TR8 polypeptide shown in FIGS. 1A-C (SEQ ID NO:2) was analyzed by a computer program (“PSORT”) (K. Nakai and M. Kanehisa, Genomics 14:897-911 (1992)), which is an expert system for predicting the cellular location of a protein based on the amino acid sequence. As part of this computational prediction of localization, the methods of McGeoch and von Heinje are incorporated. The analysis by the PSORT program predicted a signal peptide cleavage site between amino acids 23 and 24 in FIGS. 1A-C (−3 and −2 in SEQ ID NO:2). Thereafter, the complete amino acid sequence was further analyzed by visual inspection, applying a simple form of the (−1, −3) rule of von Heine. von Heinje, supra. Thus, potential leader sequences for the TR8 protein shown in SEQ ID NO:2 were predicted to consist of amino acid residues −25 to −5, or −25 to −3, or −25 to −1 in SEQ ID NO:2, while the shortest predicted mature TR8 protein corresponding to the longer potential leader consists of amino acid residues 1 to 590 for the TR8 protein shown in SEQ ID NO:2.

As one of ordinary skill would appreciate, however, due to the possibilities of sequencing errors, as well as the variability of cleavage sites for leaders in different known proteins, the TR8 receptor polypeptide encoded by the cDNA of ATCC Deposit Number 97956 comprises about 615 amino acids, but may be anywhere in therange of 605 to 625 amino acids; and the longest predicted leader sequence of this protein is about 25 amino acids, but the actual leader may be anywhere in the range of about 15 to about 35 amino acids.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) shown in FIGS. 1A-C (SEQ ID NO: 1); DNA molecules comprising the coding sequence for the mature TR8 receptor shown in FIGS. 1A-C (SEQ ID NO:2) (about the last 590 amino acids); and DNA molecules which comprise a sequence substantially different from those described above but which, due to the degeneracy of the genetic code, still encode the TR8 receptor protein shown in FIGS. 1A-C (SEQ ID NO:2). Of course, the genetic code is well known in the art. Thus, it would be routine for one skilled in the art to generate such degenerate variants.

In another aspect, the invention provides isolated nucleic acid molecules encoding the TR8 polypeptide having the amino acid sequence encoded by the cDNA clone contained in the plasmid deposited as ATCC Deposit No. 97956 on Mar. 13, 1997. In a further embodiment, these nucleic acid molecules will encode a mature polypeptide or the full-length polypeptide lacking the N-terminal methionine. The invention further provides isolated nucleic acid molecules having the nucleotide sequences shown in FIGS. 1A-C (SEQ ID NO:1), the nucleotide sequence of the cDNA contained in the above-described deposited clone; or nucleic acid molecules having a sequence complementary to one of the above sequences. Such isolated molecules, particularly DNA molecules, are useful as probes for gene mapping, by in situ hybridization with chromosomes, and for detecting expression of the TR8 receptor genes of the present invention in human tissue, for instance, by Northern blot analysis.

The present invention is further directed to fragments of the isolated nucleic acid molecules described herein. By a fragment of an isolated nucleic acid molecule having the nucleotide sequence of the deposited cDNA, the nucleotide sequence shown in FIGS. 1A-C (SEQ ID NO:1), or complementary strand thereto, is intended fragments at least about 15 nt, and more preferably at least about 20 nt, still more preferably at least about 30 nt, and even more preferably, at least about 40, 50, 100, 150, 200, 250, 300, 400, or 500 nt in length. These fragments have numerous uses which include, but are not limited to, diagnostic probes and primers as discussed herein. Of course, larger fragments, such as those of 500-1500 nt in length are also useful according to the present invention as are fragments corresponding to most, if not all, of the nucleotide sequences of the deposited cDNA or as shown in FIGS. 1A-C (SEQ ID NO:1). By a fragment at least 20 nt in length, for example, is intended fragments which include 20 or more contiguous bases from the nucleotide sequence of the deposited cDNA or the nucleotide sequence as shown in FIGS. 1A-C (SEQ ID NO: 1).

Representative examples of TR8 polynucleotide fragments of the invention include, for example, fragments that comprise, or alternatively, consist of a sequence from about nucleotide 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001-2050, or 2051 to the end of SEQ ID NO: 1, or the complementary DNA strand thereto, or the cDNA contained in the deposited clone. In this context “about” includes the particularly recited ranges, larger or smaller by several (5, 4, 3, 2, or 1) nucleotides, at either terminus or at both termini. Preferably, these fragments encode a polypeptide which demonstrates a functional activity. By a polypeptide demonstrating “functional activity” is meant, a polypeptide capable of displaying one or more known functional activities associated with a complete or mature TR8 polypeptide. Such functional activities include, but are not limited to, biological activity, antigenicity [ability to bind (or compete with a TR8 polypeptide for binding) to an anti-TR8 antibody], immunogenicity (ability to generate antibody which binds to a TR8 polypeptide), and ability to bind to a receptor or ligand for a TR8 polypeptide.

Preferred nucleic acid fragments of the present invention include nucleic acid molecules encoding one or more TR8 receptor protein domains. In particular embodiments, such nucleic acid fragments include nucleic acid molecules encoding: a polypeptide comprising the TR8 receptor protein of FIGS. 1A-C (SEQ ID NO:2) extracellular domain (predicted to constitute amino acid residues from about 26 to about 211 in FIGS. 1A-C (amino acid residues 1 to 186 in SEQ ID NO:2)); a polypeptide comprising the TR8 receptor transmembrane domain (amino acid residues 212 to 230 in FIGS. 1A-C (amino acid residues 187 to 205 in SEQ ID NO:2)); a polypeptide comprising the TR8 receptor intracellular domain (predicted to constitute amino acid residues from about 231 to about 615 in FIGS. 1A-C (amino acid residues 206 to 590 in SEQ ID NO:2)); and a polypeptide comprising the TR8 receptor protein of FIGS. 1A-C (SEQ ID NO:2) extracellular and intracellular domains with all or part of the transmembrane domain deleted.

As above with the leader sequence, the amino acid residues constituting the extracellular, transmembrane and intracellular domains have been predicted by computer analysis. Thus, as one of ordinary skill would appreciate, the amino acid residues constituting these domains may vary slightly (e.g., by about 1 to about 15 amino acid residues) depending on the criteria used to define each domain.

Preferred nucleic acid fragments of the present invention also include nucleic acid molecules encoding epitope-bearing portions of the TR8 receptor proteins. In particular, such nucleic acid fragments of the present invention include nucleic acid molecules encoding: a polypeptide comprising amino acid residues from about 35 to about 90 in FIGS. 1A-C (amino acid residues 10 to 65 in SEQ ID NO:2); a polypeptide comprising amino acid residues from about 107 to about 210 in FIGS. 1A-C (amino acid residues 82 to 185 in SEQ ID NO:2); a polypeptide comprising amino acid residues from about 236 to about 282 in FIGS. 1A-C (amino acid residues 211 to 257 in SEQ ID NO:2); a polypeptide comprising amino acid residues from about 292 to about 537 in FIGS. 1A-C (amino acid residues 267 to 512 in SEQ ID NO:2); and a polypeptide comprising amino acid residues from about 556 to about 615 in FIGS. 1A-C (amino acid residues 531 to 590 in SEQ ID NO:2). The inventors have determined that the above polypeptide fragments are antigenic regions of the TR8 receptors. Methods for determining other such epitope-bearing portions of the TR8 proteins are described in detail below.

In another aspect, the invention provides isolated nucleic acid molecules comprising polynucleotides which hybridizes, preferably under stringent hybridization conditions, to a portion of the polynucleotide of one of the nucleic acid molecules of the invention described herein, for instance, the cDNA clone contained in ATCC Deposit 97956. By “stringent hybridization conditions” is intended overnight incubation at 42° C. in a solution comprising: 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70, or 80-150 nt, or the entire length of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotide (e.g., the deposited cDNA or the nucleotide sequence as shown in FIGS. 1A-C (SEQ ID NO:1).

Of course, a polynucleotide which hybridizes only to a poly A sequence (such as the 3′ terminal poly(A) tract of a cDNA sequence), or to a complementary stretch of T (or U) residues, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention which encode TR8 polypeptides may include, but are not limited to, those encoding the amino acid sequences of the mature polypeptides, by itself; the coding sequence for the mature polypeptides and additional sequences, such as those encoding the potential leader or signal peptide sequences, such as pre-, or pro- or prepro-protein sequences; the coding sequence of the mature polypeptides, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities. Thus, the sequence encoding the polypeptides may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (Qiagen, Inc.), among others, many of which are commercially available. As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37:767 (1984). As discussed below, other such fusion proteins include the TR8 receptors fused to IgG-Fc at the N- or C-terminus.

The present invention further relates to variants of the nucleic acid molecules of the present invention, which encode portions, analogs or derivatives of the TR8 receptors. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques.

Such variants include those produced by nucleotide substitutions, deletions or additions, which may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the TR8 receptors or portions thereof. Also especially preferred in this regard are conservative substitutions.

Further embodiments of the invention include isolated nucleic acid molecules comprising a polynucleotide having a nucleotide sequence at least 90% identical, and more preferably at least 95%, 96%, 97%, 98% or 99% identical to (a) a nucleotide sequence encoding the TR8 polypeptide having the complete amino acid sequence shown in FIGS. 1A-C (amino acid residues −25 to 590 in SEQ ID NO:2); (b) a nucleotide encoding the complete amino sequence shown in FIGS. 1A-C but lacking the N-terminal methionine (i.e., amino acids −24 to 590 in SEQ ID NO:2); (c) a nucleotide sequence encoding the predicted mature TR8 receptors (full-length polypeptide with any attending leader sequence removed) comprising the amino acid sequence at positions from about 26 to about 615 in FIGS. 1A-C (amino acid residues 1 to 590 in SEQ ID NO:2); (d) a nucleotide sequence encoding the TR8 polypeptide having the complete amino acid sequence including the leader encoded by the cDNA clone contained in ATCC Deposit Number 97956; (e) a nucleotide sequence encoding the mature TR8 receptor having the amino acid sequences encoded by the cDNA clone contained in ATCC Deposit Number 97956; (f) a nucleotide sequence encoding the TR8 receptor extracellular domain; (g) a nucleotide sequence encoding the TR8 receptor transmembrane domain; (h) a nucleotide sequence encoding the TR8 receptor intracellular domain; (i) a nucleotide sequence encoding the TR8 receptor extracellular and intracellular domains with all or part of the transmembrane domain deleted; and (j) a nucleotide sequence complementary to any of the nucleotide sequences in (a), (b), (c), (d), (e), (f), (g), (h), or (i).

By a polynucleotide having a nucleotide sequence at least, for example, 95% “identical” to a reference nucleotide sequence encoding a TR8 polypeptide is intended that the nucleotide sequence of the polynucleotide is identical to the reference sequence except that the polynucleotide sequence may include up to five point mutations per each 100 nucleotides of the reference nucleotide sequence encoding the TR8 receptors. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. The reference (query) sequence may be the entire TR8 nucleotide sequence shown in FIGS. 1A-C (SEQ ID NO:1) or any fragment as described herein.

As a practical matter, whether any particular nucleic acid molecule is at least 90%, 95%, 96%, 97%, 98% or 99% identical to, for instance, the nucleotide sequence shown in FIGS. 1A-C (SEQ ID NO:1) or to the nucleotides sequence of the deposited cDNA clone encoding that protein can be determined conventionally using known computer programs such as the Bestfit program (Wisconsin Sequence Analysis Package, Version 8 for Unix, Genetics Computer Group, University Research Park, 575 Science Drive, Madison, Wis. 53711. Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. (Comp. App. Biosci. 6:237-245 (1990)). Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter. According to this embodiment, if the subject sequence is shorter than the query sequence because of 5′ or 3′ deletions, not because of internal deletions, a manual correction is made to the results to take into consideration the fact that the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. A determination of whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of this embodiment. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are made for the purposes of this embodiment.

The present application is directed to nucleic acid molecules at least 90%, 95%, 96%, 97%, 98% or 99% identical to the nucleic acid sequences shown in FIGS. 1A-C (SEQ ID NO: 1) or to the nucleic acid sequence of the deposited cDNA, irrespective of whether they encode a polypeptide having TR8 receptor activity. This is because even where a particular nucleic acid molecule does not encode a polypeptide having TR8 receptor activity, one of skill in the art would still know how to use the nucleic acid molecule, for instance, as a hybridization probe or a polymerase chain reaction (PCR) primer. Uses of the nucleic acid molecules of the present invention that do not encode a polypeptide having TR8 receptor activity include, inter alia, (1) isolating a TR8 receptor gene or allelic or splice variants thereof in a cDNA library; (2) in situ hybridization (e.g., “FISH”) to metaphase chromosomal spreads to provide precise chromosomal location of a TR8 receptor gene, as described in Verma et al., Human Chromosomes: A Manual of Basic Techniques, Pergamon Press, New York (1988); and (3) Northern Blot analysis for detecting TR8 receptor mRNA expression in specific tissues.

Preferred, however, are nucleic acid molecules having sequences at least 90%, 95%, 96%, 97%, 98% or 99% identical to the nucleic acid sequence shown in FIGS. 1A-C (SEQ ID NO:1), the nucleic acid sequence of the deposited cDNA, or fragments thereof, which do, in fact, encode a polypeptide having TR8 receptor activity. By “a polypeptide having TR8 receptor activity” is intended polypeptides exhibiting activity similar, but not necessarily identical, to an activity of the TR8 receptors of the present invention (either the full-length protein, the splice variants, or, preferably, the mature protein), as measured in a particular biological assay. For example, TR8 receptor activity can be measured by determining the ability of a polypeptide-Fc fusion protein to inhibit lymphocyte proliferation. TR8 receptor activity may also be measured by determining the ability of a polypeptide, such as cognate ligand which is free or expressed on a cell surface, to confer proliferatory activity in intact cells expressing the receptor.

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNA or the nucleic acid sequence shown in FIGS. 1A-C (SEQ ID NO: 1), or fragments thereof, will encode polypeptides “having TR8 receptor activity.” In fact, since degenerate variants of any of these nucleotide sequences all encode the same polypeptide, in many instances, this will be clear to the skilled artisan even without performing the above described comparison assay. It will be further recognized in the art that, for such nucleic acid molecules that are not degenerate variants, a reasonable number will also encode a polypeptide having TR8 protein activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in Bowie et al., “Deciphering the Message in Protein Sequences: Tolerance to Amino Acid Substitutions,” Science 247:1306-1310 (1990), wherein the authors indicate that proteins are surprisingly tolerant of amino acid substitutions.

Vectors and Host Cells

The present invention also relates to vectors which include the isolated DNA molecules of the present invention, host cells which are genetically engineered with the recombinant vectors, and the production of TR8 polypeptides or fragments thereof using these host cells or host cells that have otherwise been genetically engineered using techniques known in the art to express a polypeptide of the invention.

The polynucleotides may be joined to a vector containing a selectable marker for propagation in a host. Generally, a plasmid vector is introduced in a precipitate, such as a calcium phosphate precipitate, or in a complex with a charged lipID. If the vector is a virus, it may be packaged in vitro using an appropriate packaging cell line and then transduced into host cells.

In one embodiment, the DNA of the invention is operatively associated with an appropriate heterologous regulatory element (e.g. promoter or enhancer), such as the phage lambda PL promoter, the E. coli lac, trp and tac promoters, the SV40 early and late promoters and promoters of retroviral LTRs, to name a few. Other suitable promoters or enhancers will be known to the skilled artisan.

In embodiments in which vectors contain expression constructs, these constructs will further contain sites for transcription initiation, termination and, in the transcribed region, a ribosome binding site for translation. The coding portion of the mature transcripts expressed by the vector expression constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate heterologous hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art.

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pTR840, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Selection of appropriate vectors and promoters for expression in a host cell is a well known procedure and the requisite techniques for expression vector construction, introduction of the vector into the host and expression in the host are routine skills in the art.

The present invention also relates to host cells containing the vector constructs discussed herein, and additionally encompasses host cells containing nucleotide sequences of the invention that are operably associated with one or more heterologous control regions (e.g., promoter and/or enhancer) using techniques known of in the art. The host cell can be a higher eukaryotic cell, such as a mammalian cell (e.g., a human derived cell), or a lower eukaryotic cell, such as a yeast cell, or the host cell can be a prokaryotic cell, such as a bacterial cell. The host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., phosphorylation, cleavage) of proteins. Appropriate cell lines can be chosen to ensure the desired modifications and processing of the foreign protein expressed.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptide may be expressed in a modified form, such as a fusion protein (comprising the polypeptide joined via a peptide bond to a heterologous protein sequence (of a different protein)), and may include not only secretion signals, but also additional heterologous functional regions. Such a fusion protein can be made by ligating polynucleotides of the invention and the desired nucleic acid sequence encoding the desired amino acid sequence to each other, by methods known in the art, in the proper reading frame, and expressing the fusion protein product by methods known in the art. Alternatively, such a fusion protein can be made by protein synthetic techniques, e.g., by use of a peptide synthesizer. Thus, for instance, a region of additional amino acids, particularly charged amino acids, may be added to the N-terminus of the polypeptide to improve stability and persistence in the host cell, during purification, or during subsequent handling and storage. Additionally, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art.

A preferred fusion protein comprises a heterologous region from immunoglobulin that is useful to solubilize proteins. For example, EP-A-O 464 533 (Canadian counterpart 2045869) discloses fusion proteins comprising various portions of constant region of immunoglobin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as, human hIL-5 receptor have been fused with Fc portions for the purpose of high-throughput screening assays to identify antagonists of hIL-5. See, D. Bennett et al., Journal of Molecular Recognition, 8:52-58 (1995) and K. Johanson et al., The Journal of Biological Chemistry. 270, No. 16:9459-9471 (1995).

TR8 receptors can be recovered and purified from recombinant cell cultures by standard methods which include, but are not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, or alternatively, may be missing the N-terminal methonine, in some cases as a result of host-mediated processes.

TR8 Polypeptides and Fragments

The invention further provides isolated TR8 polypeptides having the amino acid sequence encoded by the deposited cDNAs, or the amino acid sequence in FIGS. 1A-C (SEQ ID NO:2) or a peptide or polypeptide comprising a portion of the above polypeptides.

The polypeptides of this invention may be membrane bound or may be in a soluble circulating form. Soluble peptides are defined by amino acid sequence wherein the sequence comprises the polypeptide sequence lacking the transmembrane domain.

The polypeptides of the present invention may exist as a membrane bound receptor having a transmembrane region and an intra- and extracellular region or they may exist in soluble form wherein the transmembrane domain is lacking. One example of such a form of the TR8 receptor is the TR8 receptor shown in FIGS. 1A-C (SEQ ID NO:2) which contains, in addition to a leader sequence, transmembrane, intracellular and extracellular domains. Thus, this form of the TR8 receptor appears to be localized in the cytoplasmic membrane of cells which express this protein.

It will be recognized in the art that some amino acid sequences of the TR8 receptors can be varied without significant effect to the structure or function of the protein. If such differences in sequence are contemplated, it should be remembered that there will be critical areas on the protein which determine activity. Thus, the invention further includes variations of the TR8 receptors which show substantial TR8 receptor activity or which include regions of TR8 proteins such as the protein portions discussed below. Such mutants include deletions, insertions, inversions, repeats, and type substitutions. As indicated above, guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Thus, the fragment, derivative or analog of the polypeptide of FIGS. 1A-C (SEQ ID NO:2), or that encoded by the deposited cDNA, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence which is employed for purification of the mature polypeptide or a proprotein sequence. Such fragments, derivatives and analogs are deemed to be within the scope of those skilled in the art from the teachings herein.

Of particular interest are substitutions of charged amino acids with another charged amino acid and with neutral or negatively charged amino acids. The latter results in proteins with reduced positive charge to improve the characteristics of the TR8 proteins. The prevention of aggregation is highly desirable. Aggregation of proteins not only results in a loss of activity but can also be problematic when preparing pharmaceutical formulations, because they can be immunogenic. (Pinckard et al., Clin Exp. Immunol. 2:331-340 (1967); Robbins et al., Diabetes 36:838-845 (1987); Cleland et al. Crit. Rev. Therapeutic Drug Carrier Systems 10:307-377 (1993)).

The replacement of amino acids can also change the selectivity of binding to cell surface receptors. Ostade et al., Nature 361:266-268 (1993) describes certain mutations resulting in selective binding of TNF-α to only one of the two known types of TNF receptors. Thus, the TR8 receptors of the present invention may include one or more amino acid substitutions, deletions or additions, either from natural mutations or human manipulation.

As indicated, changes are preferably of aminor nature, such as conservative amino acid substitutions that do not significantly affect the folding or activity of the protein (see Table 1).

TABLE 1
CONSERVATIVE AMINO ACID SUBSTITUTIONS.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

In specific embodiments, the number of substitutions, additions or deletions in the amino acid sequence of FIGS. 1A-C and/or any of the polypeptide fragments described herein (e.g., the extracellular domain or intracellular domain) is 75, 70, 60, 50, 40, 35, 30, 25, 20, 15, 10, 9, 8, 7, 6, 5, 4, 3, 2, 1 or 20-10, 5-10, 1-5,1-3 or 1-2.

Amino acids in the TR8 polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro, or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al., Science 255:306-312 (1992)).

The polypeptides of the present invention are preferably provided in an isolated form. By “isolated polypeptide”, is intended a polypeptide removed from its native environment. Thus, a polypeptide produced and contained within a recombinant host cell would be considered “isolated” for purposes of the present invention. Also intended as an “isolated polypeptide” are polypeptides that have been purified, partially or substantially, from a recombinant host. For example, recombinantly produced versions of the TR8 receptors can be substantially purified by the one-step method described in Smith and Johnson, Gene 67:31-40 (1988).

The polypeptides of the present invention also include the polypeptide encoded by the deposited cDNA including the leader; the polypeptide encoded by the deposited the cDNA minus the leader (i.e., the mature protein); the polypeptide of FIGS. 1A-C (SEQ ID NO:2) including the leader; the polypeptides of FIGS. 1A-C (SEQ ID NO:2) including the leader but minus the N-terminal methionine; the polypeptide of FIGS. 1A-C (SEQ ID NO:2) minus the leader; the extracellular domain, the transmembrane domain, and the intracellular domain of the TR8 receptor shown in FIGS. 1A-C (SEQ ID NO:2); and polypeptides which are at least 80% identical, more preferably at least 90% or 95% identical, still more preferably at least 96%, 97%, 98% or 99% identical to the polypeptides described above, and also include portions of such polypeptides with at least 30 amino acids and more preferably at least 50 amino acids.

By a polypeptide having an amino acid sequence at least, for example, 95% “identical” to a reference amino acid sequence of a TR8 polypeptide is intended that the amino acid sequence of the polypeptide is identical to the reference sequence except that the polypeptide sequence may include up to five amino acid alterations per each 100 amino acids of the reference amino acid of a TR8 receptor. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in FIGS. 1A-C (SEQ ID NO:2), the amino acid sequence encoded by the deposited cDNA clone, or fragments thereof, can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. (Comp. App. Biosci. 6:237-245 (1990)). Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty-0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made for the purposes of this embodiment.

The polypeptides of the present invention have uses which include, but are not limited to, molecular weight marker on SDS-PAGE gels or on molecular sieve gel filtration columns using methods well known to those of skill in the art.

For many proteins, including the extracellular domain of a membrane associated protein or the mature form(s) of a secreted protein, it is known in the art that one or more amino acids may be deleted from the N-terminus or C-terminus without substantial loss of biological function. However, even if deletion of one or more amino acids from the N-terminus or C-terminus of a protein results in modification or loss of one or more biological functions of the protein, other TR8 functional activities may still be retained. For example, in many instances, the ability of the shortened protein to induce and/or bind to antibodies which recognize TR8 (preferably antibodies that bind specifically to TR8) will retained irrespective of the size or location of the deletion. Whether a particular polypeptide lacking N-terminal and/or C-terminal residues of a complete protein retains such immunologic activities can readily be determined by routine methods described herein and otherwise known in the art.

In one embodiment, the present invention further provides polypeptides having one or more residues deleted from the amino terminus of the amino acid sequence of the TR8 polypeptide depicted in FIGS. 1A-C (SEQ ID NO:2) or encoded by the cDNA of the deposited clone. Particularly, in one embodiment, N-terminal deletions of the TR8 polypeptide can be described by the general formula m to 590, where m is a number from −24 to 589 corresponding to the position of amino acid identified in SEQ ID NO:2 and preferably, corresponds to one of the N-terminal amino acid residues identified in the N-terminal deletions specified herein. In specific embodiments, N-terminal deletions of the TR8 polypeptide of the invention comprise, or preferably consist of, amino acid residues: L-2 to A-590; Q-3 to A-590; I-4 to A-590; A-5 to A-590; P-6 to A-590; P-7 to A-590; C-8 to A-590; T-9 to A-590; S-10 to A-590; E-11 to A-590; K-12 to A-590; H-13 to A-590; Y-14 to A-590; E-15 to A-590; H-16 to A-590; L-17 to A-590; G-18 to A-590; R-19 to A-590; C-20 to A-590; C-21 to A-590; N-22 to A-590; K-23 to A-590; C-24 to A-590; E-25 to A-590; P-26 to A-590; G-27 to A-590; K-28 to A-590; Y-29 to A-590; M-30 to A-590; S-31 to A-590; S-32 to A-590; K-33 to A-590; C-34 to A-590; T-35 to A-590; T-36 to A-590; T-37 to A-590; S-38 to A-590; D-39 to A-590; S-40 to A-590; V-41 to A-590; C-42 to A-590; L-43 to A-590; P-44 to A-590; C-45 to A-590; G-46 to A-590; P-47 to A-590; D-48 to A-590; E-49 to A-590; Y-50 to A-590; L-51 to A-590; D-52 to A-590; S-53 to A-590; W-54 to A-590; N-55 to A-590; E-56 to A-590; E-57 to A-590; D-58 to A-590; K-59 to A-590; C-60 to A-590; L-61 to A-590; L-62 to A-590; H-63 to A-590; K-64 to A-590; V-65 to A-590; C-66 to A-590; D-67 to A-590; T-68 to A-590; G-69 to A-590; K-70 to A-590; A-71 to A-590; L-72 to A-590; V-73 to A-590; A-74 to A-590; V-75 to A-590; V-76 to A-590; A-77 to A-590; G-78 to A-590; N-79 to A-590; S-80 to A-590; T-81 to A-590; T-82 to A-590; P-83 to A-590; R-84 to A-590; R-85 to A-590; C-86 to A-590; A-87 to A-590; C-88 to A-590; T-89 to A-590; A-90 to A-590; G-91 to A-590; Y-92 to A-590; H-93 to A-590; W-94 to A-590; S-95 to A-590; Q-96 to A-590; D-97 to A-590; C-98 to A-590; E-99 to A-590; C-100 to A-590; C-101 to A-590; R-102 to A-590; R-103 to A-590; N-104 to A-590; T-105 to A-590; E-106 to A-590; C-107 to A-590; A-108 to A-590; P-109 to A-590; G-110 to A-590; L-111 to A-590; G-112 to A-590; A-113 to A-590; Q-114 to A-590; H-115 to A-590; P-116 to A-590; L-117 to A-590; Q-118 to A-590; L-119 to A-590; N-120 to A-590; K-121 to A-590; D-122 to A-590; T-123 to A-590; V-124 to A-590; C-125 to A-590; K-126 to A-590; P-127 to A-590; C-128 to A-590; L-129 to A-590; A-130 to A-590; G-131 to A-590; Y-132 to A-590; F-133 to A-590; S-134 to A-590; D-135 to A-590; A-136 to A-590; F-137 to A-590; S-138 to A-590; S-139 to A-590; T-140 to A-590; D-141 to A-590; K-142 to A-590; C-143 to A-590; R-144 to A-590; P-145 to A-590; W-146 to A-590; T-147 to A-590; N-148 to A-590; C-149 to A-590; T-150 to A-590; F-151 to A-590; L-152 to A-590; G-153 to A-590; K-154 to A-590; R-155 to A-590; V-156 to A-590; E-157 to A-590; H-158 to A-590; H-159 to A-590; G-160 to A-590; T-161 to A-590; E-162 to A-590; K-163 to A-590; S-164 to A-590; D-165 to A-590; V-166 to A-590; V-167 to A-590; C-168 to A-590; S-169 to A-590; S-170 to A-590; S-171 to A-590; L-172 to A-590; P-173 to A-590; A-174 to A-590; R-175 to A-590; K-176 to A-590; P-177 to A-590; P-178 to A-590; N-179 to A-590; E-180 to A-590; P-181 to A-590; H-182 to A-590; V-183 to A-590; Y-184 to A-590; L-185 to A-590; P-186 to A-590; G-187 to A-590; L-188 to A-590; I-189 to A-590; I-190 to A-590; L-191 to A-590; L-192 to A-590; L-193 to A-590; F-194 to A-590; A-195 to A-590; S-196 to A-590; V-197 to A-590; A-198 to A-590; L-199 to A-590; V-200 to A-590; A-201 to A-590; A-202 to A-590; I-203 to A-590; I-204 to A-590; F-205 to A-590; G-206 to A-590; V-207 to A-590; C-208 to A-590; Y-209 to A-590; R-210 to A-590; K-211 to A-590; K-212 to A-590; G-213 to A-590; K-214 to A-590; A-215 to A-590; L-216 to A-590; T-217 to A-590; A-218 to A-590; N-219 to A-590; L-220 to A-590; W-221 to A-590; H-222 to A-590; W-223 to A-590; I-224 to A-590; N-225 to A-590; E-226 to A-590; A-227 to A-590; C-228 to A-590; G-229 to A-590; R-230 to A-590; L-231 to A-590; S-232 to A-590; G-233 to A-590; D-234 to A-590; K-235 to A-590; E-236 to A-590; S-237 to A-590; S-238 to A-590; G-239 to A-590; D-240 to A-590; S-241 to A-590; C-242 to A-590; V-243 to A-590; S-244 to A-590; T-245 to A-590; H-246 to A-590; T-247 to A-590; A-248 to A-590; N-249 to A-590; F-250 to A-590; G-251 to A-590; Q-252 to A-590; Q-253 to A-590; G-254 to A-590; A-255 to A-590; C-256 to A-590; E-257 to A-590; G-258 to A-590; V-259 to A-590; L-260 to A-590; L-261 to A-590; L-262 to A-590; T-263 to A-590; L-264 to A-590; E-265 to A-590; E-266 to A-590; K-267 to A-590; T-268 to A-590; F-269 to A-590; P-270 to A-590; E-271 to A-590; D-272 to A-590; M-273 to A-590; C-274 to A-590; Y-275 to A-590; P-276 to A-590; D-277 to A-590; Q-278 to A-590; G-279 to A-590; G-280 to A-590; V-281 to A-590; C-282 to A-590; Q-283 to A-590; G-284 to A-590; T-285 to A-590; C-286 to A-590; V-287 to A-590; G-288 to A-590; G-289 to A-590; G-290 to A-590; P-291 to A-590; Y-292 to A-590; A-293 to A-590; Q-294 to A-590; G-295 to A-590; E-296 to A-590; D-297 to A-590; A-298 to A-590; R-299 to A-590; M-300 to A-590; L-301 to A-590; S-302 to A-590; L-303 to A-590; V-304 to A-590; S-305 to A-590; K-306 to A-590; T-307 to A-590; E-308 to A-590; I-309 to A-590; E-310 to A-590; E-311 to A-590; D-312 to A-590; S-313 to A-590; F-314 to A-590; R-315 to A-590; Q-316 to A-590; M-317 to A-590; P-318 to A-590; T-319 to A-590; E-320 to A-590; D-321 to A-590; E-322 to A-590; Y-323 to A-590; M-324 to A-590; D-325 to A-590; R-326 to A-590; P-327 to A-590; S-328 to A-590; Q-329 to A-590; P-330 to A-590; T-331 to A-590; D-332 to A-590; Q-333 to A-590; I-334 to A-590; L-335 to A-590; F-336 to A-590; L-337 to A-590; T-338 to A-590; E-339 to A-590; P-340 to A-590; G-341 to A-590; S-342 to A-590; K-343 to A-590; S-344 to A-590; T-345 to A-590; P-346 to A-590; P-347 to A-590; F-348 to A-590; S-349 to A-590; E-350 to A-590; P-351 to A-590; L-352 to A-590; E-353 to A-590; V-354 to A-590; G-355 to A-590; E-356 to A-590; N-357 to A-590; D-358 to A-590; S-359 to A-590; L-360 to A-590; S-361 to A-590; Q-362 to A-590; C-363 to A-590; F-364 to A-590; T-365 to A-590; G-366 to A-590; T-367 to A-590; Q-368 to A-590; S-369 to A-590; T-370 to A-590; V-371 to A-590; G-372 to A-590; S-373 to A-590; E-374 to A-590; S-375 to A-590; C-376 to A-590; N-377 to A-590; C-378 to A-590; T-379 to A-590; E-380 to A-590; P-381 to A-590; L-382 to A-590; C-383 to A-590; R-384 to A-590; T-385 to A-590; D-386 to A-590; W-387 to A-590; T-388 to A-590; P-389 to A-590; M-390 to A-590; S-391 to A-590; S-392 to A-590; E-393 to A-590; N-394 to A-590; Y-395 to A-590; L-396 to A-590; Q-397 to A-590; K-398 to A-590; E-399 to A-590; V-400 to A-590; D-401 to A-590; S-402 to A-590; G-403 to A-590; H-404 to A-590; C-405 to A-590; P-406 to A-590; H-407 to A-590; W-408 to A-590; A-409 to A-590; A-410 to A-590; S-411 to A-590; P-412 to A-590; S-413 to A-590; P-414 to A-590; N-415 to A-590; W-416 to A-590; A-417 to A-590; D-418 to A-590; V-419 to A-590; C-420 to A-590; T-421 to A-590; G-422 to A-590; C-423 to A-590; R-424 to A-590; N-425 to A-590; P-426 to A-590; P-427 to A-590; G-428 to A-590; E-429 to A-590; D-430 to A-590; C-431 to A-590; E-432 to A-590; P-433 to A-590; L-434 to A-590; V-435 to A-590; G-436 to A-590; S-437 to A-590; P-438 to A-590; K-439 to A-590; R-440 to A-590; G-441 to A-590; P-442 to A-590; L-443 to A-590; P-444 to A-590; Q-445 to A-590; C-446 to A-590; A-447 to A-590; Y-448 to A-590; G-449 to A-590; M-450 to A-590; G-451 to A-590; L-452 to A-590; P-453 to A-590; P-454 to A-590; E-455 to A-590; E-456 to A-590; E-457 to A-590; A-458 to A-590; S-459 to A-590; R-460 to A-590; T-461 to A-590; E-462 to A-590; A-463 to A-590; R-464 to A-590; D-465 to A-590; Q-466 to A-590; P-467 to A-590; E-468 to A-590; D-469 to A-590; G-470 to A-590; A-471 to A-590; D-472 to A-590; G-473 to A-590; R-474 to A-590; L-475 to A-590; P-476 to A-590; S-477 to A-590; S-478 to A-590; A-479 to A-590; R-480 to A-590; A-481 to A-590; G-482 to A-590; A-483 to A-590; G-484 to A-590; S-485 to A-590; G-486 to A-590; I-487 to A-590; S-488 to A-590; P-489 to A-590; G-490 to A-590; G-491 to A-590; Q-492 to A-590; S-493 to A-590; P-494 to A-590; A-495 to A-590; S-496 to A-590; G-497 to A-590; N-498 to A-590; V-499 to A-590; T-500 to A-590; G-501 to A-590; N-502 to A-590; S-503 to A-590; N-504 to A-590; S-505 to A-590; T-506 to A-590; F-507 to A-590; I-508 to A-590; S-509 to A-590; S-510 to A-590; G-511 to A-590; Q-512 to A-590; V-513 to A-590; M-514 to A-590; N-515 to A-590; F-516 to A-590; K-517 to A-590; G-518 to A-590; D-519 to A-590; I-520 to A-590; I--521 to A-590; V-522 to A-590; V-523 to A-590; Y-524 to A-590; V-525 to A-590; S-526 to A-590; Q-527 to A-590; T-528 to A-590; S-529 to A-590; Q-530 to A-590; E-531 to A-590; G-532 to A-590; A-533 to A-590; A-534 to A-590; A-535 to A-590; A-536 to A-590; A-537 to A-590; E-538 to A-590; P-539 to A-590; M-540 to A-590; G-541 to A-590; R-542 to A-590; P-543 to A-590; V-544 to A-590; Q-545 to A-590; E-546 to A-590; E-547 to A-590; T-548 to A-590; L-549 to A-590; A-550 to A-590; R-551 to A-590; R-552 to A-590; D-553 to A-590; S-554 to A-590; F-555 to A-590; A-556 to A-590; G-557 to A-590; N-558 to A-590; G-559 to A-590; P-560 to A-590; R-561 to A-590; F-562 to A-590; P-563 to A-590; D-564 to A-590; P-565 to A-590; C-566 to A-590; G-567 to A-590; G-568 to A-590; P-569 to A-590; E-570 to A-590; G-571 to A-590; L-572 to A-590; R-573 to A-590; E-574 to A-590; P-575 to A-590; E-576 to A-590; K-577 to A-590; A-578 to A-590; S-579 to A-590; R-580 to A-590; P-581 to A-590; V-582 to A-590; Q-583 to A-590; E-584 to A-590; Q-585 to A-590; of SEQ ID NO:2. Polynucleotides encoding these polypeptides are also encompassed by the invention.

In another embodiment, N-terminal deletions of the TR8 polypeptide can be described by the general formula m to 184 where m is a number from −24 to 183 corresponding to the amino acid sequence identified in SEQ ID NO:2. In specific embodiments, N terminal deletions of the TR8 of the invention comprise, or preferably, consist of, amino acids residues: L-2 to Y-184; Q-3 to Y-184; I-4 to Y-184; A-5 to Y-184; P-6 to Y-184; P-7 to Y-184; C-8 to Y-184; T-9 to Y-184; S-10 to Y-184; E-11 to Y-184; K-12 to Y-184; H-13 to Y-184; Y-14 to Y-184; E-15 to Y-184; H-16 to Y-184; L-17 to Y-184; G-18 to Y-184; R-19 to Y-184; C-20 to Y-184; C-21 to Y-184; N-22 to Y-184; K-23 to Y-184; C-24 to Y-184; E-25 to Y-184; P-26 to Y-184; G-27 to Y-184; K-28 to Y-184; Y-29 to Y-184; M-30 to Y-184; S-31 to Y-184; S-32 to Y-184; K-33 to Y-184; C-34 to Y-184; T-35 to Y-184; T-36 to Y-184; T-37 to Y-184; S-38 to Y-184; D-39 to Y-184; S-40 to Y-184; V-41 to Y-184; C-42 to Y-184; L-43 to Y-184; P-44 to Y-184; C-45 to Y-184; G-46 to Y-184; P-47 to Y-184; D-48 to Y-184; E-49 to Y-184; Y-50 to Y-184; L-51 to Y-184; D-52 to Y-184; S-53 to Y-184; W-54 to Y-184; N-55 to Y-184; E-56 to Y-184; E-57 to Y-184; D-58 to Y-184; K-59 to Y-184; C-60 to Y-184; L-61 to Y-184; L-62 to Y-184; H-63 to Y-184; K-64 to Y-184; V-65 to Y-184; C-66 to Y-184; D-67 to Y-184; T-68 to Y-184; G-69 to Y-184; K-70 to Y-184; A-71 to Y-184; L-72 to Y-184; V-73 to Y-184; A-74 to Y-184; V-75 to Y-184; V-76 to Y-184; A-77 to Y-184; G-78 to Y-184; N-79 to Y-184; S-80 to Y-184; T-81 to Y-184; T-82 to Y-184; P-83 to Y-184; R-84 to Y-184; R-85 to Y-184; C-86 to Y-184; A-87 to Y-184; C-88 to Y-184; T-89 to Y-184; A-90 to Y-184; G-91 to Y-184; Y-92 to Y-184; H-93 to Y-184; W-94 to Y-184; S-95 to Y-184; Q-96 to Y-184; D-97 to Y-184; C-98 to Y-184; E-99 to Y-184; C-100 to Y-184; C-101 to Y-184; R-102 to Y-184; R-103 to Y-184; N-104 to Y-184; T-105 to Y-184; E-106 to Y-184; C-107 to Y-184; A-108 to Y-184; P-109 to Y-184; G-110 to Y-184; L-111 to Y-184; G-112 to Y-184; A-113 to Y-184; Q-114 to Y-184; H-115 to Y-184; P-116 to Y-184; L-117 to Y-184; Q-118 to Y-184; L-119 to Y-184; N-120 to Y-184; K-121 to Y-184; D-122 to Y-184; T-123 to Y-184; V-124 to Y-184; C-125 to Y-184; K-126 to Y-184; P-127 to Y-184; C-128 to Y-184; L-129 to Y-184; A-130 to Y-184; G-131 to Y-184; Y-132 to Y-184; F-133 to Y-184; S-134 to Y-184; D-135 to Y-184; A-136 to Y-184; F-137 to Y-184; S-138 to Y-184; S-139 to Y-184; T-140 to Y-184; D-141 to Y-184; K-142 to Y-184; C-143 to Y-184; R-144 to Y-184; P-145 to Y-184; W-146 to Y-184; T-147 to Y-184; N-148 to Y-184; C-149 to Y-184; T-150 to Y-184; F-151 to Y-184; L-152 to Y-184; G-153 to Y-184; K-154 to Y-184; R-155 to Y-184; V-156 to Y-184; E-157 to Y-184; H-158 to Y-184; H-159 to Y-184; G-160 to Y-184; T-161 to Y-184; E-162 to Y-184; K-163 to Y-184; S-164 to Y-184; D-165 to Y-184; V-166 to Y-184; V-167 to Y-184; C-168 to Y-184; S-169 to Y-184; S-170 to Y-184; S-171 to Y-184; L-172 to Y-184; P-173 to Y-184; A-174 to Y-184; R-175 to Y-184; K-176 to Y-184; P-177 to Y-184; P-178 to Y-184; N-179 to Y-184; of SEQ ID NO:2. Polynucleotides encoding these polypeptides are also encompassed by the invention.

Further embodiments of the invention are directed to C-terminal deletions of the TR8 polypeptide described by the general formula 1 to n, where n is a number from 2-589 corresponding to the position of amino acid residue identified in SEQ ID NO:2 and preferably, corresponds to one of the C-terminal amino acid residues identified in the C-terminal deletions specified herein. In specific embodiments, C terminal deletions of the TR8 polypeptide of the invention comprise or preferably, consist of, amino acid residues: A-1 to K-589; A-1 to A-588; A-1 to G-587; A-1 to G-586; A-1 to Q-585; A-1 to E-584; A-1 to Q-583; A-1 to V-582; A-1 to P-581; A-1 to R-580; A-1 to S-579; A-1 to A-578; A-1 to K-577; A-1 to E-576; A-1 to P-575; A-1 to E-574; A-1 to R-573; A-1 to L-572; A-1 to G-571; A-1 to E-570; A-1 to P-569; A-1 to G-568; A-1 to G-567; A-1 to C-566; A-1 to P-565; A-1 to D-564; A-1 to P-563; A-1 to F-562; A-1 to R-561; A-1 to P-560; A-1 to G-559; A-1 to N-558; A-1 to G-557; A-1 to A-556; A-1 to F-555; A-1 to S-554; A-1 to D-553; A-1 to R-552; A-1 to R-551; A-1 to A-550; A-1 to L-549; A-1 to T-548; A-1 to E-547; A-1 to E-546; A-1 to Q-545; A-1 to V-544; A-1 to P-543; A-1 to R-542; A-1 to G-541; A-1 to M-540; A-1 to P-539; A-1 to E-538; A-1 to A-537; A-1 to A-536; A-1 to A-535; A-1 to A-534; A-1 to A-533; A-1 to G-532; A-1 to E-531; A-1 to Q-530; A-1 to S-529; A-1 to T-528; A-1 to Q-527; A-1 to S-526; A-1 to V-525; A-1 to Y-524; A-1 to V-523; A-1 to V-522; A-1 to I-521; A-1 to I-520; A-1 to D-519; A-1 to G-518; A-1 to K-517; A-1 to F-516; A-1 to N-515; A-1 to M-514; A-1 to V-513; A-1 to -512; A-1 to G-511; A-1 to S-510; A-1 to S-509; A-1 to I-508; A-1 to F-507; A-1 to T-506; A-1 to S-505; A-1 to N-504; A-1 to S-503; A-1 to N-502; A-1 to G-501; A-1 to T-500; A-1 to V-499; A-1 to N-498; A-1 to G-497; A-1 to S-496; A-1 to A-495; A-1 to P-494; A-1 to S-493; A-1 to Q-492; A-1 to G-491; A-1 to G-490; A-1 to P-489; A-1 to S-488; A-1 to I-487; A-1 to G-486; A-1 to S-485; A-1 to G-484; A-1 to A-483; A-1 to G-482; A-1 to A-481; A-1 to R-480; A-to 1 A-479; A-1 to S-478; A-1 to S-477; A-1 to P-476; A-1 to L-475; A-1 to R-474; A-1 to G-473; A-1 to D-472; A-1 to A-471; A-1 to G-470; A-1 to D-469; A-1 to E-468; A-1 to P-467; A-1 to Q-466; A-to 1 D-465; A-1 to R-464; A-1 to A-463; A-1 to E-462; A-1 to T-461; A-1 to R-460; A-1 to S-459; A-1 to A-458; A-1 to E-457; A-1 to E-456; A-1 to E-455; A-1 to P-454; A-1 to P-453; A-1 to L-452; A-to 1 G-451; A-1 to M-450; A-1 to G-449; A-1 to Y-448; A-1 to A-447; A-1 to C-446; A-1 to Q-445; A-1 to P-444; A-1 to L-443; A-1 to P-442; A-1 to G-441; A-1 to R-440; A-1 to K-439; A-1 to P-438; A-to 1 S-437; A-1 to G-436; A-1 to V-435; A-1 to L-434; A-! to P-433; A-1 to E-432; A-1 to C-431; A-1 to D-430; A-1 to E-429; A-1 to G-428; A-1 to P-427; A-1 to P-426; A-1 to N-425; A-1 to R-424; A-to 1 C-423; A-1 to G-422; A-1 to T-421; A-1 to C-420; A-1 to V-419; A-1 to D-418; A-1 to A-417; A-1 to W-416; A-1 to N-415; A-1 to P-414; A-1 to S-413; A-1 to P-412; A-1 to S-411; A-1 to A-410; A-to 1 A-409; A-1 to W-408; A-1 to H-407; A-1 to P-406; A-1 to C-405; A-1 to H-404; A-1 to G-403; A-1 to S-402; A-1 to D-401; A-1 to V-400; A-1 to E-399; A-1 to K-398; A-1 to Q-397; A-1 to L-396; A-to 1 Y-395; A-1 to N-394; A-1 to E-393; A-1 to S-392; A-1 to S-391; A-1 to M-390; A-1 to P-389; A-1 to T-388; A-1 to W-387; A-1 to D-386; A-1 to T-385; A-1 to R-384; A-1 to C-383; A-1 to L-382; A-to 1 P-381; A-1 to E-380; A-1 to T-379; A-1 to C-378; A-1 to N-377; A-1 to C-376; A-1 to S-375; A-1 to E-374; A-1 to S-373; A-1 to G-372; A-1 to V-371; A-1 to T-370; A-1 to S-369; A-1 to Q-368; A-to 1 T-367; A-1 to G-366; A-1 to T-365; A-1 to F-364; A-1 to C-363; A-1 to Q-362; A-1 to S-361; A-1 to L-360; A-1 to S-359; A-1 to D-358; A-1 to N-357; A-1 to E-356; A-1 to G-355; A-1 to V-354; A-to 1 E-353; A-1 to L-352; A-1 to P-351; A-1 to E-350; A-1 to S-349; A-1 to F-348; A-1 to P-347; A-1 to P-346; A-1 to T-345; A-1 to S-344; A-1 to K-343; A-1 to S-342; A-1 to G-341; A-1 to P-340; A-1 to E-339; A-1 to T-338; A-1 to L-337; A-1 to F-336; A-1 to L-335; A-1 to L-334; A-1 to Q-333; A-1 to D-332; A-1 to T-331; A-1 to P-330; A-1 to Q-329; A-1 to S-328; A-1 to P-327; A-1 to R-326; A-1 to D-325; A-1 to M-324; A-1 to Y-323; A-1 to E-322; A-1 to D-321; A-1 to E-320; A-1 to T-319; A-1 to P-318; A-1 to M-317; A-1 to Q-316; A-1 to R-315; A-1 to F-314; A-1 to S-313; A-1 to D-312; A-to 1 E-311; A-1 to E-310; A-1 to I-309; A-1 to E-308; A-1 to T-307; A-to 1 K-306; A-1 to S-5; A-1 to V-304; A-1 to L-303; A-1 to S-302; A-1 to L-301; A-1 to M-300; A-1 to R-299; A-1 to A-298; A-1 to D-297; A-1 to E-296; A-1 to G-295; A-1 to Q-294; A-1 to A-293; A-to 1 Y-292; A-1 to P-291; A-1 to G-290; A-1 to G-289; A-1 to G-288; A-1 to V-287; A-1 to C-286; A-1 to T-285; A-1 to G-284; A-1 to Q-283; A-1 to C-282; A-1 to V-281; A-1 to G-280; A-1 to G-279; A-to 1 Q-278; A-1 to D-277; A-1 to P-276; A-1 to Y-275; A-1 to C-274; A-1 to M-273; A-1 to D-272; A-1 to E-271; A-1 to P-270; A-1 to F-269; A-1 to T-268; A-1 to K-267; A-1 to E-266; A-1 to E-265; A-1 to L-264; A-1 to T-263; A-1 to L-262; A-1 to L-261; A-1 to L-260; A-1 to V-259; A-1 to G-258; A-1 to E-257; A-1 to C-256; A-1 to A-255; A-1 to G-254; A-1 to Q-253; A-1 to Q-252; A-1 to G-251; A-to 1 F-250; A-1 to N-249; A-1 to A-248; A-1 to T-247; A-1 to H-246; A-1 to T-245; A-1 to S-244; A-1 to V-243; A-1 to C-242; A-1 to S-241; A-1 to D-240; A-1 to G-239; A-1 to S-238; A-1 to S-237; A-to 1 E-236; A-1 to K-235; A-1 to D-234; A-1 to G-233; A-1 to S-232; A-1 to L-231; A-1 to R-230; A-1 to G-229; A-1 to C-228; A-1 to A-227; A-1 to E-226; A-1 to N-225; A-1 to I-224; A-1 to W-223; A-to 1H-222; A-1 to W-221; A-1 to L-220; A-1 to N-219; A-1 to A-218; A-1 to T-217; A-1 to L-216; A-1 to A-215; A-1 to K-214; A-1 to G-213; A-1 to K-212; A-1 to K-211; A-1 to R-210; A-1 to Y-209; A-to 1 C-208; A-1 to V-207; A-1 to G-206; A-1 to F-205; A-1 to I-204; A-1 to I-203; A-1 to A-202; A-1 to A-201; A-1 to V-200; A-1 to L-199; A-1 to A-198; A-1 to V-197; A-1 to S-196; A-1 to A-195; A-to 1 F-194; A-1 to L-193; A-1 to L-192; A-1 to L-191; A-1 to I-190; A-to 1I-189; A-1 to L-188; A-1 to G-187; A-1 to P-186; A-1 to L-185; A-1 to Y-184; A-1 to V-183; A-1 to H-182; A-1 to P-181; A-1 to E-180; A-1 to N-179; A-1 to P-178; A-1 to P-177; A-1 to K-176; A-to 1 R-175; A-1 to A-174; A-1 to P-173; A-1 to L-172; A-1 to S-171; A-1 to S-170; A-1 to S-169; A-1 to C-168; A-1 to V-167; A-1 to V-166; A-1 to D-165; A-1 to S-164; A-1 to K-163; A-1 to E-162; A-to 1 T-161; A-1 to G-160; A-1 to H-159; A-1 to H-158; A-1 to E-157; A-1 to V-156; A-1 to R-155; A-1 to K-154; A-1 to G-153; A-1 to L-152; A-1 to F-151; A-1 to T-150; A-1 to C-149; A-1 to N-148; A-to 1 T-147; A-1 to W-146; A-1 to P-145; A-1 to R-144; A-1 to C-143; A-1 to K-142; A-1 to D-141; A-1 to T-140; A-1 to S-139; A-1 to S-138; A-1 to F-137; A-1 to A-136; A-1 to D-135; A-1 to S-134; A-1 to F-133; A-1 to Y-132; A-1 to G-131; A-1 to A-130; A-1 to L-129; A-1 to C-128; A-1 to P-127; A-1 to K-126; A-1 to C-125; A-1 to V-124; A-1 to T-123; A-1 to D-122; A-1 to K-121; A-1 to N-120; A-to 1 L-119; A-1 to Q-118; A-1 to L-117; A-1 to P-116; A-1 to H-115; A-1 to Q-114; A-1 to A-113; A-1 to G-112; A-1 to L-111; A-1 to G-110; A-1 to P-109; A-1 to A-108; A-1 to C-107; A-1 to E-106; A-to 1 T-105; A-1 to N-104; A-1 to R-103; A-1 to R-102; A-1 to C-101; A-1 to C-100; A-1 to E-99; A-1 to C-98; A-1 to D-97; A-1 to Q-96; A-1 to S-95; A-1 to W-94; A-1 to H-93; A-1 to Y-92; A-1 to G-91; A-1 to A-90; A-1 to T-89; A-1 to C-88; A-1 to A-87; A-1 to C-86; A-to 1 R-85; A-1 to R-84; A-1 to P-83; A-1 to T-82; A-1 to T-81; A-1 to S-80; A-1 to N-79; A-1 to G-78; A-1 to A-77; A-1 to V-76; A-1 to V-75; A-1 to A-74; A-1 to V-73; A-1 to L-72; A-1 to A-71; A-1 to K-70; A-1 to G-69; A-1 to T-68; A-1 to D-67; A-1 to C-66; A-1 to V-65; A-1 to K-64; A-1 to H-63; A-1 to L-62; A-1 to L-61; A-1 to C-60; A-1 to K-59; A-1 to D-58; A-1 to E-57; A-1 to E-56; A-1 to N-55; A-1 to W-54; A-1 to S-53; A-1 to D-52; A-1 to L-51; A-1 to Y-50; A-1 to E-49; A-1 to D-48; A-1 to P-47; A-1 to G-46; A-1 to C-45; A-1 to P-44; A-1 to L-43; A-1 to C-42; A-1 to V-41; A-1 to S-40; A-1 to D-39; A-1 to S-38; A-1 to T-37; A-1 to T-36; A-1 to T-35; A-1 to C-34; A-1 to K-33; A-1 to S-32; A-1 to S-31; A-1 to M-30; A-1 to Y-29; A-1 to K-28; A-1 to G-27; A-1 to P-26; A-1 to E-25; A-1 to C-24; A-1 to K-23; A-1 to N-22; A-1 to C-21; A-1 to C-20; A-1 to R-19; A-1 to G-18; A-1 to L-17; A-1 to H-16; A-1 to E-15; A-1 to Y-14; A-1 to H-13; A-1 to K-12; A-1 to E-11; A-1 to S-10; A-1 to T-9; A-1 to C-8; A-1 to P-7; A-1 to P-6; of SEQ ID NO:2. Polynucleotides encoding these polypeptides are also encompassed by the invention.

Further embodiments of the invention are directed to polypeptide fragments comprising, or preferably, consisting of, amino acids described by the general formula m to n, where m and n correspond to any one of the amino acid residues specified above for these symbols, respectively.

Polypeptide fragments of the present invention include polypeptides comprising an amino acid sequence contained in SEQ ID NO:2, encoded by the cDNA contained in the deposited clone, or encoded by nucleic acids which hybridize (e.g., under stringent hybridization conditions) to the nucleotide sequence contained in the deposited clone, or shown in FIGS. 1A-C (SEQ ID NO:1) or the complementary strand thereto. Protein fragments may be “free-standing,” or comprised within a larger polypeptide of which the fragment forms a part or region, most preferably as a single continuous region. Representative examples of polypeptide fragments of the invention, include, for example, fragments that comprise or alternatively, consist of from about amino acid residues −25 to 1, 1 to 20, 21 to 40, 41 to 60, 61 to 80, 81 to 100, 102 to 120, 121 to 140, 141 to 160, 161 to 180, 181 to 200, 201 to 220, 221 to 240, 241 to 260, 261 to 280, 281 to 310, 311 to 350, 351 to 400, 401 to 450, 451 to 500, 551 to 600, or 601 to the end of the coding region of SEQ ID NO:2. Moreover, polypeptide fragments can be at least about 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

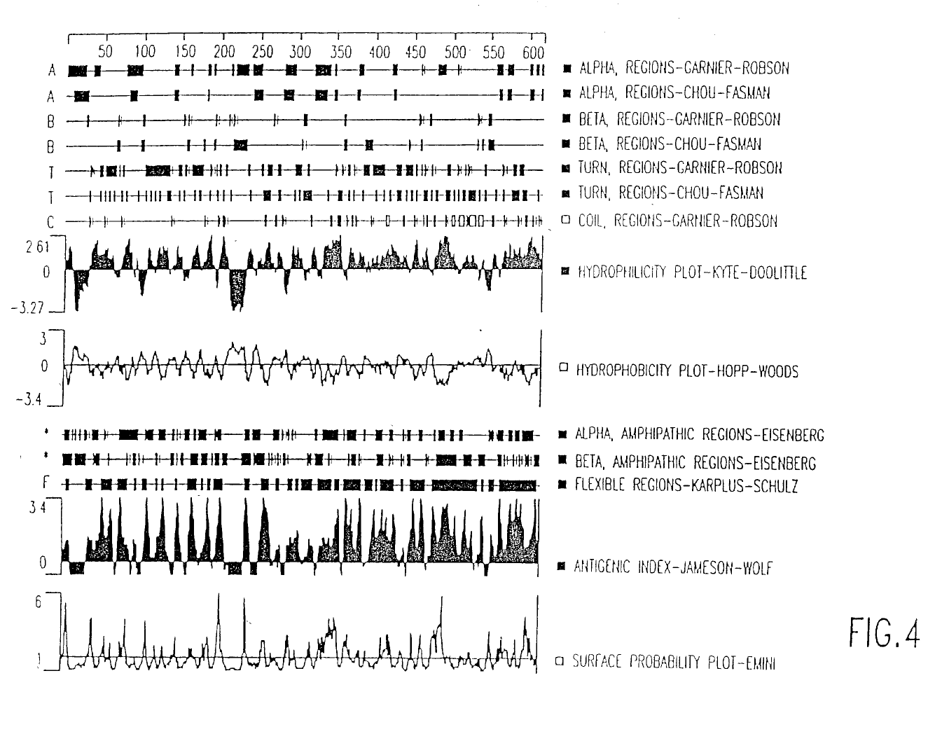

Among the especially preferred fragments of the invention are fragments characterized by structural or functional attributes of TR8. Such fragments include amino acid residues that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, surface forming regions, and high antigenic index regions (i.e., having an antigenic index of or equal to greater than 0.7, as identified using the default parameters of the Jameson-Wolf program) of TR8. Certain preferred regions are those set out in FIG. 4 and include, but are not limited to, regions of the aforementioned types identified by analysis of the amino acid sequence depicted in FIGS. 1A-C, such preferred regions include; Garnier-Robson predicted alpha-regions, beta-regions, turn-regions, and coil-regions; Chou-Fasman predicted alpha-regions, beta-regions, turn-regions, and coil-regions; Kyte-Doolittle predicted hydrophilic and hydrophobic regions; Eisenberg alpha and beta amphipathic regions; Emini surface-forming regions; and Jameson-Wolf high antigenic index regions, as predicted using the default parameters of these computer programs. Polynucleotides encoding these polypeptides are also encompassed by the invention.

In specific embodiments, polypeptide fragments of the invention comprise, or alternatively consist of, amino acid residues: 20 to 34, 20 to 60, 20 to 66, 20 to 168, 45 to 60, 66 to 86, 86 to 106, 88 to 100, 101 to 125, 128 to 143, 143 to 149, 149 to 168, 41 to 46, 123 to 128, and/or 138 to 150 as depicted in SEQ ID NO:2.

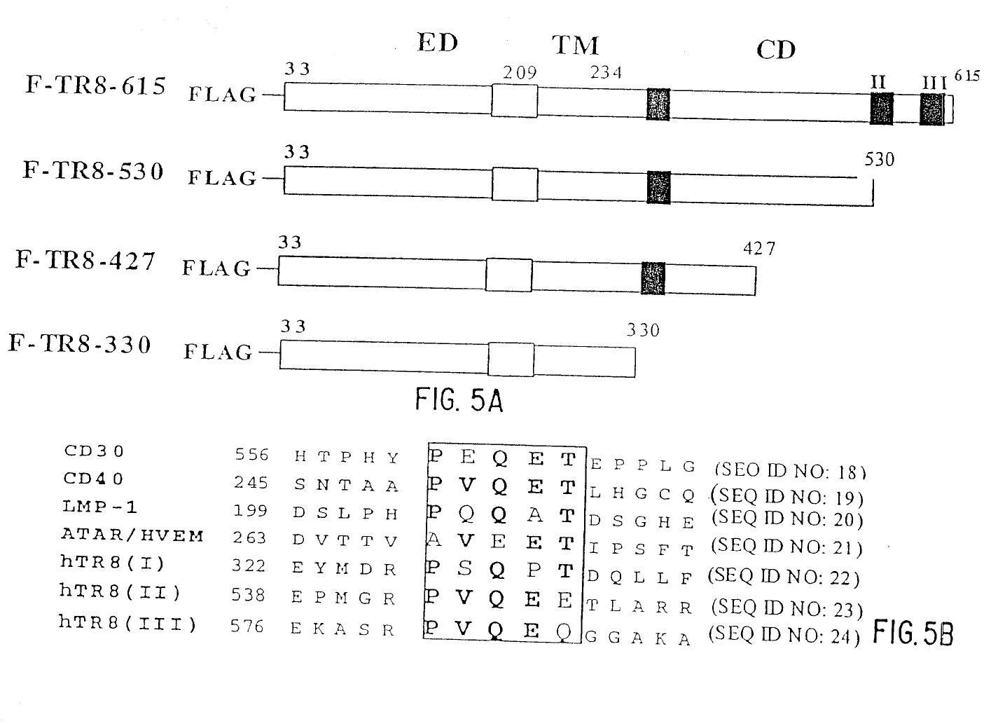

In additional specific embodiments, polypeptide fragments of the invention comprise one or more of the three potential conserved (boxed) TRAF binding domains in TR8 (See, FIG. 5B)

In other embodiments, the fragments or polypeptides of the invention (i.e., those described herein) are not larger than 570, 550, 525, 500, 475, 450, 400, 425, 390, 380, 375, 350, 336, 334, 331, 300, 275, 250, 225, 200, 185, 175, 170, 165, 160, 155, 150, 145, 140, 135, 130, 125, 120, 115, 110, 105, 100, 90, 80, 75, 60, 50, 40, 30, or 25 amino acid residues in length.

In another aspect, the invention provides peptides or polypeptides comprising epitope-bearing portions of the polypeptides of the invention. The epitopes of these polypeptide portions are an immunogenic or antigenic epitopes of the polypeptides described herein. An “immunogenic epitope” is defined as a part of a protein that elicits an antibody response when the whole protein is the immunogen. On the other hand, a region of a protein molecule to which an antibody can bind is defined as an “antigenic epitope.”

The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Learner, R. A. (1983). Antibodies that react with predetermined sites on proteins. Science 219:660-666. Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals.

Antigenic epitope-bearing peptides and polypeptides of the invention are therefore useful to raise antibodies, including monoclonal antibodies, that bind specifically to a polypeptide of the invention. See, for instance, Wilson et al., Cell 37:767-778 (1984) at 777. Antigenic epitope-bearing peptides and polypeptides of the invention preferably contain a sequence of at least seven, more preferably at least nine and most preferably between at least about 15 to about 30 amino acids contained within the amino acid sequence of a polypeptide of the invention.

Non-limiting examples of antigenic polypeptides or peptides that can be used to generate TR8 receptor-specific antibodies include: a polypeptide comprising amino acid residues from about 35 to about 90 in FIGS. 1A-C (amino acid residues 10 to 65 in SEQ ID NO:2); a polypeptide comprising amino acid residues from about 107 to about 210 in FIGS. 1A-C (amino acid residues 82 to 185 in SEQ ID NO:2); a polypeptide comprising amino acid residues from about 236 to about 282 in FIGS. 1A-C (amino acid residues 211 to 257 in SEQ ID NO:2); a polypeptide comprising amino acid residues from about 292 to about 537 in FIGS. 1A-C (amino acid residues 267 to 512 in SEQ ID NO:2); and a polypeptide comprising amino acid residues from about 556 to about 615 in FIGS. 1A-C (amino acid residues 531 to 590 in SEQ ID NO:2). As indicated above, the inventors have determined that the above polypeptide fragments are antigenic regions of the TR8 receptor proteins.

The epitope-bearing peptides and polypeptides of the invention may be produced by any conventional means. Houghten, R. A. (1985) General method for the rapid solid-phase synthesis of large numbers of peptides: specificity of antigen-antibody interaction at the level of individual amino acids. Proc. Natl. Acad. Sci. USA 82:5131-5135. This “Simultaneous Multiple Peptide Synthesis (SMPS)” process is further described in U.S. Pat. No. 4,631,211 to Houghten et al. (1986).

As one of skill in the art will appreciate, TR8 polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric TR8 receptor proteins or protein fragments alone (Fountoulakis et al., J Biochem 270:3958-3964 (1995)).

Detection of Disease States

The TNF-family ligands induce various cellular responses by binding to TNF-family receptors, including the TR8 receptors of the present invention. TNF-β, a potent ligand of the TNF receptor proteins, is known to be involved in a number of biological processes including lymphocyte development, tumor necrosis, induction of an antiviral state, activation of polymorphonuclear leukocytes, induction of class I major histocompatibility complex antigens on endothelial cells, induction of adhesion molecules on endothelium and growth hormone stimulation (Ruddle and Homer, Prog. Allergy, 40:162-182 (1988)). TNF-α, also a ligand of the TNF receptor proteins, has been reported to have a role in the rapid necrosis of tumors, immunostimulation, autoimmune disease, graft rejection, producing an anti-viral response, septic shock, cerebral malaria, cytotoxicity, protection against deleterious effects of ionizing radiation produced during a course of chemotherapy, such as denaturation of enzymes, lipid peroxidation and DNA damage (Nata et al, J. Immunol. 136(7):2483 (1987); Porter, Tibtech 9:158-162 (1991)), growth regulation, vascular endothelium effects and metabolic effects. TNF-α also triggers endothelial cells to secrete various factors, including PAI-1, IL-1, GM-CSF and IL-6 to promote cell proliferation. In addition, TNF-α up-regulates various cell adhesion molecules such as E-Selectin, ICAM-1 and VCAM-1. TNF-α and the Fas ligand have also been shown to induce programmed cell death.

Cells which express the TR8 polypeptides and are believed to have a potent cellular response to TR8 receptor ligands include dendritic cells. In addition, Northern blots revealed an approximately 4 kb mRNA observed most abundantly in colon, to a lesser extent in small intestine, lymph node and pancreas, barely detectable in spleen, fetal liver, lung prostate, thymus, testis and ovary, which was not observed in peripheral blood leukocytes, bone marrow, heart, brain, liver, skeletal muscle or kidney. By “a cellular response to a TNF-family ligand” is intended any genotypic, phenotypic, and/or morphologic change to a cell, cell line, tissue, tissue culture or patient that is induced by a TNF-family ligand. As indicated, such cellular responses include not only normal physiological responses to TNF-family ligands, but also diseases associated with increased cell proliferation or the inhibition of increased cell proliferation, such as by the inhibition of apoptosis. Apoptosis-programmed cell death-is a physiological mechanism involved in the deletion of peripheral T lymphocytes of the immune system, and its dysregulation can lead to a number of different pathogenic processes (Ameisen, J. C., AIDS 8:1197-1213 (1994); Krammer et al., Curr. Opin. Immunol. 6:279-289 (1994)).

It is believed that certain tissues in mammals with specific disease states associated with aberrant cell survival express significantly altered levels of the TR8 receptor protein and mRNA encoding the TR8 receptor protein when compared to a corresponding “standard” mammal, i.e., a mammal of the same species not having the disease state. Further, since some forms of this protein are secreted, it is believed that enhanced levels of the TR8 receptor protein can be detected in certain body fluids (e.g., sera, plasma, urine, and spinal fluid) from mammals with the disease state when compared to sera from mammals of the same species not having the disease state. Thus, the invention provides a diagnostic method useful during diagnosis of disease states, which involves assaying the expression level of the gene encoding the TR8 receptor protein in mammalian cells or body fluid and comparing the gene expression level with a standard TR8 receptor gene expression level, whereby an increase or decrease in the gene expression level over the standard is indicative of certain disease states associated with aberrant cell survival.

Where diagnosis of a disease state involving the TR8 receptors of the present invention has already been made according to conventional methods, the present invention is useful as a prognostic indicator, whereby patients exhibiting significantly aberrant TR8 receptor gene expression will experience a worse clinical outcome relative to patients expressing the gene at a lower level.

By “assaying the expression level of the gene encoding the TR8 receptor protein” is intended qualitatively or quantitatively measuring or estimating the level of the TR8 receptor protein or the level of the mRNA encoding the TR8 receptor protein in a first biological sample either directly (e.g., by determining or estimating absolute protein level or mRNA level) or relatively (e.g., by comparing to the TR8 receptor protein level or mRNA level in a second biological sample).

Preferably, the TR8 receptor protein level or mRNA level in the first biological sample is measured or estimated and compared to a standard TR8 receptor protein level or mRNA level, the standard being taken from a second biological sample obtained from an individual not having the disease state. As will be appreciated in the art, once a standard TR8 receptor protein level or mRNA level is known, it can be used repeatedly as a standard for comparison.

By “biological sample” is intended any biological sample obtained from an individual, cell line, tissue culture, or other source which contains TR8 receptor protein or mRNA. Biological samples include mammalian body fluids (such as sera, plasma, urine, synovial fluid and spinal fluid) which contain secreted mature TR8 receptor protein, and thymus, prostate, heart, placenta, muscle, liver, spleen, lung, kidney and other tissues. Methods for obtaining tissue biopsies and body fluids from mammals are well known in the art. Where the biological sample is to include mRNA, a tissue biopsy is the preferred source.

Diseases associated with increased cell survival, or the inhibition of apoptosis, include cancers (such as follicular lymphomas, carcinomas with p53 mutations, and hormone-dependent tumors); autoimmune disorders (such as systemic lupus erythematosus and immune-related glomerulonephritis rheumatoid arthritis) and viral infections (such as herpes viruses, pox viruses and adenoviruses), information graft v. host disease, acute graft rejection, and chronic graft rejection. Diseases associated with decreased cell survival, or increased apoptosis, include AIDS; neurodegenerative disorders (such as Alzheimer's disease, Parkinson's disease, Amyotrophic lateral sclerosis, Retinitis pigmentosa, Cerebellar degeneration); myelodysplastic syndromes (such as aplastic anemia), ischemic injury (such as that caused by myocardial infarction, stroke and reperfusion injury), toxin-induced liver disease (such as that caused by alcohol), septic shock, cachexia and anorexia.

Assays available to detect levels of soluble receptors are well known to those of skill in the art, for example, radioimmunoassays, competitive-binding assays, Western blot analysis, and preferably an ELISA assay may be employed.

TR8 receptor-protein specific antibodies can be raised against the intact TR8 receptor protein or an antigenic polypeptide fragment thereof, which may presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (niAb) is meant to include intact molecules as well as antibody fragments (such as, for example, Fab Fab (ab′) fragments) which are capable of specifically binding to TR8 receptor protein. Fab and F(ab′) fragments lack the Fc fragment of intact antibody, clear more rapidly from the circulation, and may have less non-specific tissue binding of an intact antibody (Wahl et al., J. Nucl. Med. 24:316-325 (1983)). Thus, these fragments are preferred.

The antibodies of the present invention may be prepared by any of a variety of methods using TR8 receptor immunogens of the present invention. Such TR8 receptor immunogens include the TR8 receptor protein shown in FIGS. 1A-C (SEQ ID NO:2) (which may or may not include a leader sequence) and polypeptide fragments of the receptor comprising the ligand binding, extracellular, transmembrane, the intracellular domains of the TR8 receptors, or any combination thereof. For example, cells expressing the TR8 receptor protein or an antigenic fragment thereof can be administered to an animal in order to induce the production of sera containing polyclonal antibodies. In a preferred method, a preparation of TR8 receptor protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or TR8 receptor protein binding fragments thereof). Such monoclonal antibodies can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., In: Monoclonal Antibodies and T-Cell Hybridomas, Elsevier, N.Y., (1981) pp. 563-681). In general, such procedures involve immunizing an animal (preferably a mouse) with a TR8 receptor protein antigen or, more preferably, with a TR8 receptor protein-expressing cell. Suitable cells can be recognized by their capacity to bind anti-TR8 receptor protein antibody. Such cells may be cultured in any suitable tissue culture medium; however, it is preferable to culture cells in Earle's modified Eagle's medium supplemented with 10% fetal bovine serum (inactivated at about 56 C), and supplemented with about 10 g/l of nonessential amino acids, about 1,000 U/ml of penicillin, and about 100 μg/ml of streptomycin. The splenocytes of such mice are extracted and fused with a suitable myeloma cell line. Any suitable mycloma cell line may be employed in accordance with the present invention; however, it is preferable to employ the parent myeloma cell line (SP₂O), available from the American Type Culture Collection, Rockville, Md. After fusion, the resulting hybridoma cells are selectively maintained in HAT medium, and then cloned by limiting dilution as described by Wands et al., (Gastroenterology 80:225-232 (1981)). The hybridoma cells obtained through such a selection are then assayed to identify clones which secrete antibodies capable of binding the TR8 receptor protein antigen.

Antibodies of the invention can be used in methods known in the art relating to the localization and activity of the polypeptide sequences of the invention, e.g., for imaging these polypeptides, measuring levels thereof in appropriate physiological samples, etc. The antibodies also have use in immunoassays and in therapeutics as agonists and antagonists of TR8.

Agonists and Antagonists of TR8 Receptor Function

In one aspect, the present invention is directed to a method for inhibiting an activity of TR8 induced by a TNF-family ligand (e.g., cell proliferation, hematopoictic development, osteoclast differentiation, and survival of dendritic cells), which involves administering to a cell which expresses a TR8 polypeptide, an effective amount of a TR8 receptor ligand, analog or an antagonist capable of decreasing TR8, receptor mediated signaling. Preferably, TR8 receptor mediated signaling is decreased to treat a disease wherein increased cell proliferation is exhibited. An antagonist can include soluble forms of the TR8 receptors and antibodies directed against the TR8 polypeptides which block TR8 receptor mediated signaling. Preferably, TR8 receptor mediated signaling is decreased to treat a disease, to decrease survival of cells, e.g., dendritic cells, or to delay or prevent bone formation (e.g., via osteoclast differentiation).

In a further aspect, the present invention is directed to a method for increasing cell proliferation induced by a TNF-family ligand, which involves administering to a cell which expresses a TR8 polypeptide an effective amount of an agonist capable of increasing TR8 receptor mediated signaling. Preferably, TR8 receptor mediated signaling is increased to treat a disease wherein decreased cell proliferation is exhibited wherein increased survival of cells (e.g., dendritic cells) is desired, or to stimulate bone formation (e.g., via osteoclast differentiation). Agonists of the present invention include monoclonal antibodies directed against the TR8 polypeptides which stimulate TR8 receptor mediated signaling. Preferably, TR8 receptor mediated signaling is increased to treat a disease.

By “agonist” is intended naturally occurring and synthetic compounds capable of enhancing cell proliferation, survival, and/or differentiation mediated by TR8 polypeptides. Such agonists include agents which increase expression of TR8 receptors or increase the sensitivity of the expressed receptor. By “antagonist” is intended naturally occurring and synthetic compounds capable of inhibiting TR8 mediated cell proliferation and differentiation. Such antagonists include agents which decrease expression of TR8 receptors or decrease the sensitivity of the expressed receptor. Whether any candidate “agonist” or “antagonist” of the present invention can enhance or inhibit cell proliferation, survival, and differentiation can be determined using art-known TNF-family ligand/receptor cellular response assays, including those described in more detail below.

One such screening technique involves the use of cells which express the receptor (for example, transfected CHO cells) in a system which measures extracellular pH changes caused by receptor activation, for example, as described in Science 246:181-296 (October 1989). For example, compounds may be contacted with a cell which expresses the receptor polypeptide of the present invention and a second messenger response, e.g., signal transduction or pH changes, may be measured to determine whether the potential compound activates or inhibits the receptor.

Another such screening technique involves introducing RNA encoding the receptor into Xenopus oocytes to transiently express the receptor. The receptor oocytes may then be contacted with the receptor ligand and a compound to be screened, followed by detection of inhibition or activation of a calcium signal in the case of screening for compounds which are thought to inhibit activation of the receptor.

Another method involves screening for compounds which inhibit activation of the receptor polypeptide of the present invention antagonists by determining inhibition of binding of labeled ligand to cells which have the receptor on the surface thereof. Such a method involves transfecting a eukaryotic cell with DNA encoding the receptor such that the cell expresses the receptor on its surface and contacting the cell with a compound in the presence of a labeled form of a known ligand. The ligand can be labeled, e.g., by radioactivity. The amount of labeled ligand bound to the receptors is measured, e.g., by measuring radioactivity of the receptors. If the compound binds to the receptor as determined by a reduction of labeled ligand which binds to the receptors, the binding of labeled ligand to the receptor is inhibited.

Soluble forms of the polypeptides of the present invention may be utilized in the ligand binding assay described above. These forms of the TR8 receptors are contacted with ligands in the extracellular medium after they are secreted. A determination is then made as to whether the secreted protein will bind to TR8 receptor ligands.

Further screening assays for agonist and antagonist of the present invention are described in Tartaglia and Goeddel, J. Biol. Chem. 267(7):4304-4307(1992).

Thus, in a further aspect, a screening method is provided for determining whether a candidate agonist or antagonist is capable of enhancing or inhibiting a cellular response to a TNF-family ligand. The method involves contacting cells which express TR8 polypeptides with a candidate compound and a TNF-family ligand, assaying a cellular response, and comparing the cellular response to a standard cellular response, the standard being assayed when contact is made with the ligand in absence of the candidate compound, whereby an increased cellular response over the standard indicates that the candidate compound is an agonist of the ligand/receptor signaling pathway and a decreased cellular response compared to the standard indicates that the candidate compound is an antagonist of the ligand/receptor signaling pathway. By “assaying a cellular response” is intended qualitatively or quantitatively measuring a cellular response to a candidate compound and/or a TNF-family ligand (e.g., determining or estimating an increase or decrease in T cell proliferation or tritiated thymidine labeling). By the invention, a cell expressing a TR8 polypeptide can be contacted with either an endogenous or exogenously administered TNF-family ligand.

In an additional aspect, a thymocyte proliferation assay may be employed to identify both ligands and potential drug candidates. For example, thymus cells are disaggregated from tissue and grown in culture medium. Incorporation of DNA precursors such as ³H-thymidine or 5-bromo-2′-deoxyuridine (BrdU) is monitored as a parameter for DNA synthesis and cellular proliferation. Cells which have incorporated BrdU into DNA can be detected using a monoclonal antibody against BrdU and measured by an enzyme or fluorochrome-conjugated second antibody. The reaction is quantitated by fluorimetry or by spectrophotometry. Two control wells and an experimental well are set up as above and TNF-β or cognate ligand is added to all wells while soluble receptor polypeptides of the present invention are added individually to the second control wells, with the experimental well containing a compound to be screened. The ability of the compound to be screened to stimulate or inhibit the above interaction may then be quantified.

Agonists according to the present invention include compounds such as, for example, TNF-family ligand peptide fragments, transforming growth factors, and neurotransmitters (such as glutamate, dopamine, N-methyl-D-aspartate). Preferred agonists include TR8 polypeptide fragments of the invention and/or polyclonal and monoclonal antibodies raised against TR8 polypeptide, or a fragment thereof. Such agonist antibodies raised against a TNF-family receptor are disclosed in Tartaglia, L. A., et al., Proc. Natl. Acad. Sci. USA 88:9292-9296 (1991); and Tartaglia, L. A., and Goeddel, D. V., J (7):4304-4307 (1992). See, also, PCT Application WO 94/09137. Further preferred agonists include chemotherapeutic drugs such as, for example, cisplatin, doxorubicin, bleomycin, cytosine arabinodide, nitrogen mustard, methotrexate and vincristine. Others include ethanol and -amyloid peptide. (Science 267:1457-1458 (1995)).

In specific embodiments, antagonists according to the present invention are nucleic acids corresponding to the sequences contained in FIGS. 1A-C, or the complementary strand thereof, and/or to nucleotide sequences contained in the deposited clone. In one embodiment, antisense sequence is generated internally by the organism, in another embodiment, the antisense sequence is separately administered (see, for example, O'Connor, J., Neurochem. 56:560 (1991). Oligodeoxynucleotides as Anitsense Inhibitors of Gene Expression, CRC Press, Boca Raton, Fla. (1988). Antisense technology can be used to control gene expression through antisense DNA or RNA, or through triple-helix formation. Antisense techniques are discussed for example, in Okano, J., Neurochem. 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research 6:3073 (1979); Cooney et al., Science 241:456 (1988); and Dervan et al., Science 251:1300 (1991). The methods are based on binding of a polynucleotide to a complementary DNA or RNA.

For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of the receptor. The antisense RNA oligonucleotide hybridizes to the mRNA in vivo and blocks translation of the mRNA molecule into receptor polypeptide.

In one embodiment, the TR8 antisense nucleic acid of the invention is produced intracellularly by transcription from an exogenous sequence. For example, a vector or a portion thereof, is transcribed, producing an antisense nucleic acid (RNA) of the invention. Such a vector would contain a sequence encoding the TR8 antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others know in the art, used for replication and expression in vertebrate cells. Expression of the sequence encoding TR8, or fragments thereof, can be by any promoter known in the art to act in vertebrate, preferably human cells. Such promoters can be inducible or constitutive. Such promoters include, but are not limited to, the SV40 early promoter region (Bernoist and Chambon, Nature 29:304-310 (1981), the promoter contained in the 3′ long terminal repeat of Rous sarcoma virus (Yamamoto et al., Cell 22:787-797 (1980), the herpes thymidine promoter (Wagner et al., Proc. Natl. Acad. Sci. U.S.A. 78:1441-1445 (1981), the regulatory sequences of the metallothionein gene (Brinster, et al., Nature 296:39-42 (1982)), etc.

The antisense nucleic acids of the invention comprise a sequence complementary to at least a portion of an RNA transcript of a TR8 gene. However, absolute complementarity, although preferred, is not required. A sequence “complementary to at least a portion of an RNA,” referred to herein, means a sequence having sufficient complementarity to be able to hybridize with the RNA, forming a stable duplex; in the case of double stranded TR8 antisense nucleic acids, a single strand of the duplex DNA may thus be tested, or triplex formation may be assayed. The ability to hybridize will depend on both the degree of complementarity and the length of the antisense nucleic acid Generally, the larger the hybridizing nucleic acid, the more base mismatches with a TR8 RNA it may contain and still form a stable duplex (or triplex as the case may be). One skilled in the art can ascertain a tolerable degree of mismatch by use of standard procedures to determine the melting point of the hybridized complex.

Potential antagonists according to the invention also include catalytic RNA, or a ribozyme (See, e.g., PCT International Publication WO 90/11364, published Oct. 4, 1990; Sarver et al, Science 247:1222-1225 (1990). While ribozymes that cleave mRNA at site specific recognition sequences can be used to destroy TR8 mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). There are numerous potential hammerhead ribozyme cleavage sites within the nucleotide sequence of TR8 (FIGS. 1A-C). Preferably, the ribozyme is engineered so that the cleavage recognition site is located near the 5′ end of the TR8 mRNA; i.e., to increase efficiency and minimize the intracellular accumulation of non-functional mRNA transcripts. DNA constructs encoding the ribozyme may be introduced into the cell in the same manner as described above for the introduction of antisense encoding DNA. Since ribozymes, unlike antisense molecules are catalytic, a lower intracellular concentration is required for efficiency.

In other embodiments, antagonists according to the present invention include soluble forms of the TR8 receptors (e.g., fragments of the TR8 receptor shown in FIGS. 1A-C that include the ligand binding domain from the extracellular region of the full length receptor). Such soluble forms of the receptor, which may be naturally occurring or synthetic, antagonize TR8 mediated signaling by competing with the cell surface bound forms of the receptor for binding to TNF-family ligands. Antagonists of the present invention also include antibodies specific for TNF-family ligands and TR8-Fc fusion proteins.

By a “TNF-family ligand” is intended naturally occurring, recombinant, and synthetic ligands that are capable of binding to a member of the TNF receptor family and inducing the ligand/receptor signaling pathway. Members of the TNF ligand family include, but are not limited to, TNF-α, lymphotoxin-α (LT-α, also known as TNF-β), LT-β (found in complex heterotrimer LT-α2-β), FasL, CD40L, CD27L, CD30L, 4-lBBL, OX40L and nerve growth factor (NGF).

TNF-α has been shown to protect mice from infection with herpes simplex virus type 1 (HSV-1). Rossol-Voth et al., J. Gen. Virol. 72:143-147 (1991). The mechanism of the protective effect of TNF-α is unknown but appears to involve neither interferons nor NK cell killing. One member of the TNFR family has been shown to mediate HSV-1 entry into cells. Montgomery et al., Eur. Cytokine Newt. 7:159 (1996). Further, antibodies specific for the extracellular domain of this TNFR block HSV-1 entry into cells. Thus, TR8 antagonists of the present invention include both TR8 amino acid sequences and antibodies capable of preventing TNFR mediated viral entry into cells. Such sequences and antibodies can function by either competing with cell surface localized TNFR for binding to virus or by directly blocking binding of virus to cell surface receptors.

Antibodies according to the present invention may be prepared by any of a variety of standard methods using TR8 receptor immunogens of the present invention. Such TR8 receptor immunogens include the TR8 receptor protein shown in FIGS. 1A-C (SEQ ID NO:2) (which may or may not include a leader sequence) and polypeptide fragments of the receptor comprising the ligand binding, extracellular, transmembrane, the intracellular domains of the TR8 receptors, or any combination thereof

Polyclonal and monoclonal antibody agonists or antagonists according to the present invention can be raised according to the methods disclosed in Tartaglia and Goeddel, J. Biol. Chem. 267(7):4304-4307(1992)); Tartaglia et al., Cell 73:213-216 (1993)), and PCT Application WO 94/09137 and are preferably specific to polypeptides of the invention having the amino acid sequence of SEQ ID NO:2. The term “antibody” (Ab) or “monoclonal antibody” (mAb) as used herein is meant to include intact molecules as well as fragments thereof (such as, for example, Fab and F(ab′) fragments) which are capable of binding an antigen. Fab, Fab′ and F(ab′) fragments lack the Fc fragment intact antibody, clear more rapidly from the circulation, and may have less non-specific tissue binding of an intact antibody (Wahl et al., J. Nucl. Med., 24:316-325 (1983)).

In a preferred method, antibodies according to the present invention are mAbs. Such mAbs can be prepared using hybridoma technology (Kohler and Millstein, Nature 256:495-497 (1975) and U.S. Pat. No. 4,376,110; Harlow et al., Antibodies: A Laboratory Manual, Cold Spring Harbor Laboratory Press, Cold Spring Harbor, N.Y., 1988; Monoclonal Antibodies and Hybridomas: A New Dimension in Biological Analyses, Plenum Press, New York, N.Y., 1980; Campbell, “Monoclonal Antibody Technology,” In: Laboratory Techniques in Biochemistry and Molecular Biology, Volume 13 (Burdon et al., eds.), Elsevier, Amsterdam (1984)).

Proteins and other compounds which bind the TR8 receptor domains are also candidate agonist and antagonist according to the present invention. Such binding compounds can be “captured” using the yeast two-hybrid system (Fields and Song, Nature 340:245-246 (1989)). A modified version of the yeast two-hybrid system has been described by Roger Brent and his colleagues (Gyuris et al., Cell 75:791-803 (1993); Zervos et al., Cell 72:223-232 (1993)). Preferably, the yeast two-hybrid system is used according to the present invention to capture compounds which bind to the ligand binding, extracellular, intracellular, and transmembrane domains of the TR8 receptors. Such compounds are good candidate agonist and antagonist of the present invention.

Using the two-hybrid assay described above, the intracellular domain of the TR8 receptor, or a portion thereof, may be used to identify cellular proteins which interact with the receptor in vivo. Such an assay may also be used to identify ligands with potential agonistic or antagonistic activity of TR8 receptor function. This screening assay has previously been used to identify protein which interact with the cytoplasmic domain of the murine TNF-R11 and led to the identification of two receptor associated proteins. Rothe, M. et al., Cell 78:681 (1994). Such proteins and amino acid sequences which bind to the cytoplasmic domain of the TR8 receptors are good candidate agonist and antagonist of the present invention.

Other screening techniques include the use of cells which express the polypeptide of the present invention (for example, transfected CHO cells) in a system which measures extracellular pH changes caused by receptor activation, for example, as described in Science, 246:181-296 (1989). In another example, potential agonists or antagonists may be contacted with a cell which expresses the polypeptide of the present invention and a second messenger response, e.g., signal transduction may be measured to determine whether the potential antagonist or agonist is effective.

The TR8 receptor agonists may be employed to stimulate ligand activities, such as inhibition of tumor growth and necrosis of certain transplantable tumors, or alternatively, the survival of certain cell types (e.g., dendritic cells). The agonists may also be employed to stimulate cellular differentiation, for example, T-cells, osteoclasts, fibroblasts and hemopoietic cell differentiation. Agonists to the TR8 receptor may also augment TR8's role in the host's defense against microorganisms and prevent related diseases (infections such as that from Listeria monocytogenes) and Chlamidiae. The agonists may also be employed to protect against the deleterious effects of ionizing radiation produced during a course of radiotherapy, such as denaturation of enzymes, lipid peroxidation, and DNA damage.

Agonists to the receptor polypeptides of the present invention may be used to augment TNF's role in host defenses against microorganisms and prevent related diseases. The agonists may also be employed to protect against the deleterious effects of ionizing radiation produced during a course of radiotherapy, such as denaturation of enzymes, lipid peroxidation, and DNA damage.

The agonists may also be employed to mediate an anti-viral response, to regulate growth, to mediate the immune response and to treat immunodeficiencies related to diseases such as HIV by increasing the rate of lymphocyte proliferation and differentiation.

Agonists to the receptor polypeptides of the present invention may additionally be used to effectuate bone growth (i.e., bone mass). Administration of such agonists can be used to treat bone fractures, defects, and disorders which result in weakened bones such as osteoporosis, osteomalacia, and age-related loss of bone mass. According to the invention, bone growth is enhanced by local and/or systemic administration of a TR8 agonist in an osteogenically effective amount (i.e., an amount which effects the formation and/or development of bone). Additionally, agonists of the invention may optionally be combined with osteogenically effective amounts of other bone growth promoting compounds, including beta-type transforming growth factors (“TGF-βs”); e.g., TGF-β1, 2, 3 and/or bone morphogenic proteins (“BMPs”; e.g., BMP-2,3,4,5,6, or 7) and osteogenic proteins, and/or parathyroid hormone. BMPs and TGF-βs may be prepared by methods known in the art (see e.g., PCT/US87/01537 and U.S. Pat. No. 4,774,332 which are incorporated herein by reference in their entirety). Alternatively, TGF-βs are available from commercial sources (R&D Systems, Minneapolis, Minn.).

The antagonists to the polypeptides of the present invention may be employed to inhibit ligand activities, such as, for example, stimulation of tumor growth and necrosis of certain transplantable tumors, and promoting the survival of certain cell types (e.g., dendritic cells). The antagonists may also be employed to inhibit cellular differentiation, such as, for example, T-cell, osteoclast, fibroblast, and hemopoictic cell differentiation. Antagonists may also be employed to treat autoimmune diseases, such as, for example, graft versus host rejection and allograft rejection, and T-cell mediated autoimmune diseases such as AIDS. It has been shown that T-cell proliferation is stimulated via a type 2 TNF receptor. Accordingly, antagonizing the receptor may prevent the proliferation of T-cells and treat T-cell mediated autoimmune diseases.

The state of immunodeficiency that defines AIDS is secondary to a decrease in the number and function of CD4⁺ T-lymphocytes. Recent reports estimate the daily loss of CD4⁺ T cells to be between 3.5×10⁷ and 2×10⁹ cells (Wei et al., Nature 373:117-122 (1995)). One cause of CD4⁺ T cell depletion in the setting of HIV infection is believed to be HIV-induced apoptosis. Indeed, HIV-induced apoptotic cell death has been demonstrated not only in vitro but also, more importantly, in infected individuals (Ameisen, J. C., AIDS 8:1197-1213 (1994); Finkel, T. H., and Banda, N. K., Curr. Opin. Immunol. 6:605-615(1995); Muro-Cacho et al., J. Immunol. 154:5555-5566 (1995)). Furthermore, apoptosis and CD4⁺ T-lyrnphocyte depletion is tightly correlated in different animal models of AIDS (Brunner et al., Nature 373:441-444 (1995); Gougeon et al., AIDS Res. Hum. Retroviruses 9:553-563 (1993)) and, apoptosis is not observed in those animal models in which viral replication does not result in AIDS (Gougeon et al., AIDS Res. Hum. Retroviruses 9:553-563 (1993)). Further data indicates that uninfected but primed or activated T lymphocytes from HIV-infected individuals undergo apoptosis after encountering the TNF-family ligand FasL. Using monocytic cell lines that result in death following HIV infection, it has been demonstrated that infection of U937 cells with HIV results in the de novo expression of FasL and that FasL mediates HIV-induced apoptosis (Badley et al., J. Virol. 70:199-206 (1996)). Further the TNF-family ligand was detectable in uninfected macrophages and its expression was upregulated following HIV infection resulting in selective killing of uninfected CD4⁺ T-lymphocytes (Badley et al., J. Virol. 70:199-206 (1996)).

In rejection of an allograft, the immune system of the recipient animal has not previously been primed to respond because the immune system for the most part is only primed by environmental antigens. Tissues from other members of the same species have not been presented in the same way that, for example, viruses and bacteria have been presented. In the case of allograft rejection, immunosuppressive regimens are designed to prevent the immune system from reaching the effector stage. However, the immune profile of xenograft rejection may resemble disease recurrence more than allograft rejection. In the case of disease recurrence, the immune system has already been activated, as evidenced by destruction of the native islet cells. Therefore, in disease recurrence the immune system is already at the effector stage. Antagonists of the present invention are able to suppress the immune response to both allografts and xenografts by decreasing the rate of TR8 mediated lymphocyte proliferation and differentiation. Such antagonists include the TR8-Fc fusion protein described in Example 5. Thus, the present invention further provides a method for suppression of immune responses.

In addition, TNF-α has been shown to prevent diabetes in strains of animals which are prone to this affliction resulting from autoimmunity. See Porter, A., Tibtech 9:158-162 (1991). Thus, agonists and antagonists of the present invention may be useful in the treatment of autoimmune diseases such as type 1 diabetes.

In addition, the role played by the TR8 receptors in cell proliferation, survival and differentiation indicates that agonist or antagonist of the present invention may be used to treat disease states involving aberrant cellular expression of these receptors. TR8 receptors may in some circumstances induce an inflammatory response, and antagonists may be useful reagents for blocking this response. Thus TR8 receptor antagonists (e.g., soluble forms of the TR8 receptors; neutralizing antibodies) may be useful for treating inflammatory diseases, such as rheumatoid arthritis, osteoarthritis, psoriasis, septicemia, and inflammatory bowel disease.

Antagonists to the TR8 receptor may also be employed to treat and/or prevent septic shock, which remains a critical clinical condition. Septic shock results from an exaggerated host response, mediated by protein factors such as TNF and IL-1, rather than from a pathogen directly. For example, lipopolysaccharides have been shown to elicit the release of TNF leading to a strong and transient increase of its serum concentration. TNF causes shock and tissue injury when administered in excessive amounts. Accordingly, it is believed that antagonists to the TR8 receptor will block the actions of TNF and treat/prevent septic shock. These antagonists may also be employed to treat meningococcemia in children which correlates with high serum levels of TNF.

Among other disorders which may be treated by the antagonists to TR8 receptors, there are included, inflammation which is mediated by TNF receptor ligands, and the bacterial infections cachexia and cerebral malaria. The TR8 receptor antagonists may also be employed to treat inflammation mediated by ligands to the receptor such as TNF.

Modes of Administration

The agonist or antagonists described herein can be administered in vitro, ex vivo, or in vivo to cells which express the receptor of the present invention. By administration of an “effective amount” of an agonist or antagonist is intended an amount of the compound that is sufficient to enhance or inhibit a cellular response to a TNF-family ligand and include polypeptides. In particular, by administration of an “effective amount” of an agonist or antagonists is intended an amount effective to enhance or inhibit TR8 receptor mediated activity. Of course, where cell proliferation and/or differentiation is to be enhanced, an agonist according to the present invention can be co-administered with a TNF-family ligand. One of ordinary skill will appreciate that effective amounts of an agonist or antagonist can be determined empirically and may be employed in pure form or in pharmaceutically acceptable salt, ester or pro-drug form. The agonist or antagonist may be administered in compositions in combination with one or more pharmaceutically acceptable excipients (i.e., carriers).

It will be understood that, when administered to a human patient, the total daily usage of the compounds and compositions of the present invention will be decided by the attending physician within the scope of sound medical judgment. The specific therapeutically effective dose level for any particular patient will depend upon factors well known in the medical arts.

As a general proposition, the total pharmaceutically effective amount of a TR8 polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the TR8 polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed.

Pharmaceutical compositions are provided comprising an agonist (including TR8 receptor polynucleotides or polypeptides of the invention) or agonist (e.g., TR8 polypeptides of the invention or antibodies thereto) of TR8 and a pharmaceutically acceptable carrier or excipient, which may be administered orally, rectally, parenterally, intracistemally, intravaginally, intraperitoneally, topically (as by powders, ointments, drops or transdermal patch), bucally, or as an oral or nasal spray, In one embodiment “pharmaceutically acceptable carrier” means a non-toxic solid, semisolid or liquid filler, diluent, encapsulating material or formulation auxiliary of any type. In a specific embodiment, “pharmaceutically acceptable” means approved by a regulatory agency of the federal or a state government or listed in the U.S. Pharmacopeia or other generally recognized pharmacopeia for use in animals, and more particularly humans. Nonlimiting examples of suitable pharmaceutical carriers according to this embodiment are provided in “Remington's Pharmaceutical Sciences” by E. W. Martin, and include sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glyceral solutions can be employed as liquid carriers, particularly for injectable solutions.

The term “parenteral” as used herein refers to modes of administration which include intravenous, intramuscular, intraperitoneal, intrastemal, subcutaneous and intraarticular injection and infusion.

Having generally described the invention, the same will be more readily understood by reference to the following examples, which are provided by way of illustration and are not intended as limiting.

### Example 1

### Expression and Purification of TR8 in E. coli

The bacterial expression vector pQE60 is used for bacterial expression in this example. (QIAGEN, Inc., 9259 Eton Avenue, Chatsworth, Calif., 91311). pQE60 encodes ampicillin antibiotic resistance (“Amp^{r}”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., supra, and suitable single restriction enzyme cleavage sites. These elements are arranged such that a DNA fragment encoding a polypeptide may be inserted in such as way as to produce that polypeptide with the six His residues (i.e., a “6×His tag”) covalently linked to the carboxyl terminus of that polypeptide. However, in this example, the polypeptide coding sequence is inserted such that translation of the six His codons is prevented and, therefore, the polypeptide is produced with no 6×His tag.

The DNA sequence encoding the desired portion of the TR8 protein lacking the hydrophobic leader sequence is amplified from the deposited cDNA clone using PCR oligonucleotide primers which anneal to the amino terminal sequences of the desired portion of the TR8 protein and to sequences in the deposited construct 3′ to the cDNA coding sequence. Additional nucleotides containing restriction sites to facilitate cloning in the pQE60 vector are added to the 5′ and 3′ sequences, respectively.

For cloning the soluble extracellular domain of the TR8 protein, the 5′ primer has the sequence:

5′CGCCCATGGCTTTGCAGATCGCTCCTC 3′ (SEQ ID NO:7) containing the underlined NcoI restriction site followed by 18 nucleotides complementary to the amino terminal coding sequence of the extracellular domain of theTR8 sequence in FIGS. 1A-C (nucleotides 124-142 of SEQ ID NO:1). One of ordinary skill in the art would appreciate, of course, that the point in the protein coding sequence where the 5′ primer begins may be varied to amplify a desired portion of the complete protein shorter or longer than the mature form. The 3′ primer for the soluble extracellular domain has the sequence:

5′CGCAAGCTTTTAGGGCAAGTAAACATG 3′ (SEQ ID NO:8) containing the underlined HindIII restriction site followed by 18 nucleotides complementary to the 3′ end of the nucleotide sequence shown in FIGS. 1A-C (nucleotides 667-681 in SEQ ID NO: 1) encoding the extracellular domain of the TR8 receptor.

The amplified TR8 DNA fragments and the vector pQE60 are digested with NcoI and HindIII and the digested DNAs are then ligated together. Insertion of the TR8 DNA into the restricted pQE60 vector places the TR8 protein coding region including its associated stop codon downstream from the IPTG-inducible promoter and in-frame with an initiating AUG. The associated stop codon prevents translation of the six histidine codons downstream of the insertion point.

The ligation mixture is transformed into competent E. coli cells using standard procedures such as those described in Sambrook et al., Molecular Cloning: a Laboratory Manual, 2nd Ed.; Cold Spring Harbor Laboratory Press, Cold Spring Harbor, N.Y. (1989). E. coli strain M15/rep4, containing multiple copies of the plasmid pREP4, which expresses the lac repressor and confers kanamycin resistance (“Kan^{r}”), is used in carrying out the illustrative example described herein. This strain, which is only one of many that are suitable for expressing TR8 protein, is available commercially from QIAGEN, Inc., supra. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. Isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 3-4 hours at 4° C. in 6 M guanidine-HCl, pH 8. The cell debris is removed by centrifugation, and the supernatant containing the TR8 is dialyzed against 50 mM Na-acetate buffer pH 6, supplemented with 200 mM NaCl. Alternatively, the protein can be successfully refolded by dialyzing it against 500 mM NaCl, 20% glycerol, 25 mM Tris/HCl pH 7.4, containing protease inhibitors. After renaturation the protein can be purified by ion exchange, hydrophobic interaction and size exclusion chromatography. Alternatively, an affinity chromatography step such as an antibody column can be used to obtain pure TR8 protein. The purified protein is stored at 4° C. or frozen at −80° C.

### Example 2

### Example 2(a)

### Cloning and Expression of a Soluble Fragment of TR8 Protein in a Baculovirus Expression System

In this example, the plasmid shuttle vector pA2 GP was used to insert the cloned DNA encoding the mature extracellular domain of the TR8 receptor protein shown in FIGS. 1A-C, lacking its naturally associated secretory signal (leader) sequence, into a baculovirus. This protein was expressed using a baculovirus leader and standard methods as described in Summers et al, A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by the secretory signal peptide (leader) of the baculovirus gp67 protein and convenient restriction sites such as BamHI, XbaI and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate viable virus that expresses the cloned polynucleotide.

Many other baculovirus vectors could be used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39.

The cDNA sequence encoding essentially the extracellular domain with leader (amino acids 1 to 211 shown in FIGS. 1A-C) of the TR8 receptor protein in the deposited clone (ATCC Deposit Number 97956) is amplified using PCR oligonucleotide primers corresponding to the relevant 5′ and 3′ sequences of the gene.

The 5′ primer for the above has the sequence: 5′ CGCGGATCCGCCATCATGGCCCCGCGCGCCCGGC 3′ (SEQ ID NO:9) containing the underlined BamHI restriction enzyme site, an efficient signal for initiation of translation in eukaryotic cells, as described by Kozak, M., J. Mol. Biol. 196:947-950 (1987), followed by 15 bases of the coding sequence of the TR8 protein shown in FIGS. 1A-C (nucleotides 49-67 in SEQ ID NO:1).

The 3′ primer has the sequence: 5′CGCGGTACCTTAGGGCAAGTAAACAT G 3′ (SEQ ID NO:10) containing the underlined Asp718 restriction sites followed by 17 nucleotides complementary to the coding sequence in FIGS. 1A-C (nucleotides 667-681 in SEQ ID NO:1).

The amplified fragment is isolated from a 1% agarose gel using a commercially available kit (“Geneclean,” BIO 101 Inc., La Jolla, Calif.). The fragment was then digested with BamHI and Asp718 and purified on a 1% agarose gel. This fragment is designated herein “F1”.

The plasmid is digested with the restriction enzymes BamHlI and Asp718 dephosphorylated using calf intestinal phosphatase. The DNA is then isolated from a 1% agarose gel using a commercially available kit (“Geneclean” BIO 101 Inc., La Jolla, Calif.). This vector DNA is designated herein “V1”.

Fragment F1 and the dephosphorylated plasmid VI are ligated together with T4 DNA ligase. E. coli HB10 cells are transformed with the ligation mixture and spread on culture plates. Other suitable E. coli hosts such as XL-1 Blue (Stratagene Cloning Systems, La Jolla, Calif.) may also be used. Bacteria are identified that contain the plasmid with the human TR8 sequences using the PCR method, in which one of the above primers is used to amplify the gene and the second primer is from well within the vector so that only those bacterial colonies containing TR8 gene fragments show amplification of the DNA. The sequence of the cloned fragment is confirmed by DNA sequencing. The plasmid is designated herein pBacTR8-T.

Five μg of pBacTR8-T is co-transfected with 1.0 μg of a commercially available linearized baculovirus DNA (“BaculoGold baculovirus DNA”, Pharmingen, San Diego, Calif.), using the lipofection method described by Felgner et al., Proc. Natl. Acad. Sci. USA 84:7413-7417 (1987). 1 μg of BaculoGold virus DNA and 5 μg of plasmid pBacTR8-T are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is rocked back and forth to mix the newly added solution. The plate is then incubated for 5 hours at 27° C. After 5 hours the transfection solution is removed from the plate and 1 ml of Grace's insect medium supplemented with 10% fetal calf serum is added. The plate is put back into an incubator and cultivation is continued at 27° C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10). After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C. The recombinant virus is called V-TR8-T.

To verify the expression of the gene used, Sf9 cells are grown in Grace's medium supplemented with 10% heat-inactivated FBS. The cells are infected with the recombinant baculovirus V-TR8-T at a multiplicity of infection (“MOI”) of about 2. Six hours later the medium is removed and replaced with SF900 II medium minus methionine and cysteine (available from Life Technologies Inc., Rockville, Md.). Forty-two hours later, 5 μCi of ³⁵S-methionine and 5 μCi ³⁵S-cysteine (available from Amersham) are added to radiolabel proteins. The cells are further incubated for 16 hours and then they are harvested by centrifugation. The proteins in the supernatant as well as the intracellular proteins are analyzed by SDS-PAGE followed by autoradiography. Microsequencing of the amino acid sequence of the amino terminus of purified protein is used to determine the amino terminal sequence of the mature protein and thus the cleavage point and length of the secretory signal peptide.

### Example 2(b)

### Cloning and Expression of the Full-Length Gene for TR8 Protein in a Baculovirus Expression System

Similarly to the cloning and expression of the truncated version of the TR8 receptor described in Example 2(a), recombinant baculoviruses were generated which express the full length TR8 receptor protein shown in FIGS. 1A-C (SEQ ID NO:2).

In this example, the plasmid shuttle vector pA2 is used to insert the cloned DNA encoding the complete protein, including its naturally associated secretary signal (leader) sequence, into a baculovirus to express the mature TR8 protein. Other attributes of the pA2 vector are as described for the pA2 GP vector used in Example 2(a).

The cDNA sequence encoding the full length TR8 protein in the deposited clone, including the AUG initiation codon and the naturally associated leader sequence shown in FIGS. 1A-C (SEQ ID NO:2), is amplified using PCR oligonucleotide primers corresponding to the 5′ and 3′ sequences of the gene. The 5′ primer could have the same sequence used in Example 2(a), above.

A suitable 3′ primer for this purpose has the sequence: 5′ CGCGGTACCCTGC GAGTTTGAGGAGTG 3′ (SEQ ID NO: 11) containing the underlined Asp718 restriction sites followed by 17 nucleotides complementary to the coding sequence in FIGS. 1A-C (nucleotides 2138-2155 in SEQ ID NO:1).

The amplified fragment is isolated and digested with restriction enzymes as described in Example 2(a) to produce plasmid pBacTR8

5 μg of pBacTR8 is co-transfected with 1 μg of BaculoGold (Pharmingen) viral DNA and 10 μl of Lipofectin (Life Technologies, Inc.) in a total volume of 200 μl serum free media. The primary viruses are harvested at 4-5 days post-infection (pi), and used in plaque assays. Plaque purified viruses are subsequently amplified and frozen, as described in Example 2(a).

For radiolabeling of expressed proteins, Sf9 cells are seeded in 12 well dishes with 2.0 ml of a cell suspension containing 0.5×10⁶ cells/ml and allowed to attach for 4 hours. Recombinant baculoviruses are used to infect the cells at an MOI of 1-2. After 4 hours, the media is replaced with 1.0 ml of serum free media depleted for methionine and cysteine (-Met/-Cys). At 3 days pi, the culture media is replaced with 0.5 ml -Met/-Cys containing 2 μCi each [³⁵S]-Met and [³⁵S]-Cys. Cells are labeled for 16 hours after which the culture media is removed and clarified by centrifugation (Supernatant). The cells are lysed in the dish by addition of 0.2 ml lysis buffer (20 mM HEPES, pH 7.9; 130 mM NaCl; 0.2 mM EDTA; 0.5 mM DTT and 0.5% vol/vol NP-40) and then diluted up to 1.0 ml with dH₂O (Cell Extract). 30 μl of each supernatant and cell extract are resolved by 15% SDS-PAGE. Protein gels are stained, destained, amplified, dried and autoradiographed. Labeled bands corresponding to the recombinant proteins are visible after 16-72 hours exposure.

### Example 3

### Cloning and Expression of TR8 in Mammalian Cells

A typical mammalian expression vector contains the promoter element, which mediates the initiation of transcription of mRNA, the protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as PSVL and PMSG (Pharmacia, Uppsala, Sweden), pRSVcat (ATCC 37152), pSV2dhfr (ATCC 37146) and pBC12MI (ATCC 67109). Mammalian host cells that could be used include, human HeLa 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded protein. The DHFR (dihydrofolate reductase) marker is useful to develop cell lines that carry several hundred or even several thousand copies of the gene of interest. Another useful selection marker is the enzyme glutamine synthase (GS) (Murphy et al., Biochem J 227:277-279 (1991); Bebbington et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

The expression vectors pC1 and pC4 contain the strong promoter (LTR) of the Rous Sarcoma Virus (Cullen et al., Molecular and Cellular Biology, 438447 (March, 1985)) plus a fragment of the CMV-enhancer (Boshart et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

### Example 3(a)

### Cloning and Expression in COS Cells

The expression plasmid, pTR8 HA, is made by cloning a cDNA encoding the soluble extracellular portion of the TR8 protein into the expression vector pcDNAI/Amp or pcDNAIII (which can be obtained from Invitrogen, Inc.).

The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin protein described by Wilson et al., Cell 37:767 (1984). The fusion of the HA tag to the target protein allows easy detection and recovery of the recombinant protein with an antibody that recognizes the HA epitope. pcDNAIII contains, in addition, the selectable neomycin marker.

A DNA fragment encoding a TR8 protein is cloned into the polylinker region of the vector so that recombinant protein expression is directed by the CMV promoter. The plasmid construction strategy is as follows. The TR8 cDNA of the deposited clone is amplified using primers that contain convenient restriction sites, much as described above for construction of vectors for expression of TR8 in E. coli. Suitable primers include the following, which are used in this example.

The 5′ primer, containing the underlined BamHI site, a Kozak sequence, an AUG start codon and 6 additional codons of the 5′ coding region of the complete TR8 has the following sequence: 5′CGCGGATCCGCCATCATGGCCCCGCGCGCCCGGC 3′ (SEQ ID NO:9).

The 3′ primer has the sequence: 5′CGCTCTAGATCAAGCGTAGTCTGGGA CGTCGTATGGGTATTAGGGCAAGTAAACATG 3′ (SEQ ID NO:12) containing the underlined XbaI restriction site followed by a stop codon, a sequence encoding a 6×his tag, and 15 nucleotides complementary to the coding sequence in FIGS. 1A-C (nucleotides 667-681 in SEQ ID NO:1).

The PCR amplified DNA fragment and the vector, pcDNAI/Amp, are digested with BamHI and XbaI and then ligated. The ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the TR8-encoding fragment.

For expression of recombinant TR8, COS cells are transfected with an expression vector, as described above, using DEAE-DEXTRAN, as described, for instance, in Sambrook et al., Molecular Cloning: a Laboratory Manual, Cold Spring Laboratory Press, Cold Spring Harbor, N.Y. (1989). Cells are incubated under conditions for expression of TR8 by the vector.

Expression of the TR8-HA fusion protein is detected by radiolabeling and immunoprecipitation, using methods described in, for example Harlow et al., Antibodies: A Laboratory Manual, 2nd Ed.; Cold Spring Harbor Laboratory Press, Cold Spring Harbor, N.Y. (1988). To this end, two days after transfection, the cells are labeled by incubation in media containing ³⁵S-cysteine for 8 hours. The cells and the media are collected, and the cells are washed and lysed with detergent-containing RIPA buffer: 150 mM NaCl, 1% NP-40, 0.1% SDS, 0.5% DOC, 50 mM TRIS, pH 7.5, as described by Wilson et al. cited above. Proteins are precipitated from the cell lysate and from the culture media using an HA-specific monoclonal antibody. The precipitated proteins then are analyzed by SDS-PAGE and autoradiography. An expression product of the expected size is seen in the cell lysate, which is not seen in negative controls.

### Example 3(b)

### Cloning and Expression in CHO Cells

The vector pC4 is used for the expression of TR8 protein. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., Kellems, R. M., Bertino, J. R., and Schimke, R. T., 1978, J. Biol. Chem. 253:1357-1370, Hamlin, J. L. and Ma, C. 1990, Biochem. et Biophys. Acta, 1097:107-143, Page, M. J. and Sydenham, M. A. 1991, Biotechnology 9:64-68). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al., Molecular and Cellular Biology, March 1985:438-447) plus a fragment isolated from the enhancer of the immediate early gene of human cytomegalovirus (CMV) (Boshart et al., Cell 41:521-530 (1985)). Downstream of the promoter are BamHI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human-actin promoter, the SV40 early or late promoters or the long terminal repeats from other retroviruses, e.g., HIV and HTLVI. Clontech's Tet-Off and Tet-On gene expression systems and similar systems can be used to express the TR8 protein in a regulated way in mammalian cells (Gossen, M., & Bujard, H. 1992, Proc. Natl. Acad. Sci. USA 89: 5547-5551). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with the restriction enzymes BamHlI and Asp718 and then dephosphorylated using calf intestinal phosphatase by procedures known in the art. The vector is then isolated from a 1% agarose gel.

The DNA sequence encoding the complete TR8 protein including its leader sequence is amplified using PCR oligonucleotide primers corresponding to the 5′ and 3′ sequences of the gene having, for instance, the same sequences as the 5′ and 3′ primers used for cloning in baculovirus pA vectors as shown in Example 2, above.

The amplified fragment is digested with the endonucleases BamHI and Asp718 and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. 5 μg of the expression plasmid pC4 is cotransfected with 0.5 μg of the plasmid pSV2-neo using lipofectin (Felgner et al., supra). The plasmid pSV2neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reverse phase HPLC analysis.

### Example 4

### Tissue distribution of TR8 mRNA Expression

Northern blot analysis is carried out to examine TR8 gene expression in human tissues, using methods described by, among others, Sambrook et al., cited above. A cDNA probe containing the entire nucleotide sequence of the TR8 protein (SEQ ID NO:1) is labeled with ³²P using the rediprime DNA labeling system (Amersham Life Science), according to manufacturer's instructions. After labeling, the probe is purified using a CHROMA SPIN-100 column (Clontech Laboratories, Inc.), according to manufacturer's protocol number PT1200-1. The purified labeled probe is then used to examine various human tissues for TR8 mRNA.

Multiple Tissue Northern (MTN) blots containing various human tissues (H) or human immune system tissues (IM) are obtained from Clontech and are examined with the labeled probe using ExpressHyb hybridization solution (Clontech) according to manufacturer's protocol number PT1190-1. Following hybridization and washing, the blots are mounted and exposed to film at −70° C. overnight, and films developed according to standard procedures.

### Example 5

### Example 5(a)

### Expression and Purification of TR8-Fc (TR8-Ig Fusion Protein) and Cleaved TR8

The putative transmembrane domain of translated TR8 receptor was determined by hydrophobicity using the method of Goldman et al. (Ann. Rev. of Biophys. Biophys. Chem. 15:321-353 (1986)) for identifying nonpolar transbilayer helices. The region upstream of this transmembrane domain, encoding the putative leader peptide and extracellular domain, is selected for the production of an Fc fusion protein. Primers are designed to PCR the corresponding coding region from the deposited clone with the addition of a BglII site, a Factor Xa protease site and an Asp718I site at the 3 end. PCR with this primer pair results in one band of the expected size. This is cloned into COSFclink to give the TR8-Fclink plasmID The PCR product is digested with EcoRI and Asp718I and ligated into the COSFclink plasmid (Johansen, et al., J. Biol. Chem. 270:9459-9471 (1995)) to produce TR8-Fclink.

COS cells are transiently transfected with TR8-Fclink and the resulting supernatant is immunoprecipitated with protein A agarose. Western blot analysis of the immunoprecipitate using goat anti-human Fc antibodies reveals a strong band consistent with the expected size for glycosylated TR8-Fc (greater than 65,940 kiD). A 15L transient COS transfection is performed and the resulting supernatant is purified. The purified protein is used to immunize mice following DNA injection for the production of mAbs.

CHO cells are transfected with TR8-Fclink to produce stable cell lines. Five lines are chosen by dot blot analysis for expansion and are adapted to shaker flasks. The line with the highest level of TR8-Fc protein expression is identified by Western blot analysis. TR8-Fc protein purified from the supernatant of this line is used for cell binding studies by flow cytometry, either as intact protein or after factor Xa cleavage and biotinylation.

### Example 5(b)

### Purification of TR8-Fc from CHO EIA Conditioned Media Followed by Cleavage and Biotinylation of TR8

Assays—Product purity through the purification is monitored on 15% Laemmli SDS-PAGE gels run under reducing and non-reducing conditions. Protein concentration was monitored by A₂₈₀ assuming extinction coefficients for the receptor and the chimera calculated from the sequences.

Protein G Chromatography of the TR8-Fc Fusion Protein—All steps described below are carried out at 4° C. 15L of CHO conditioned media (CM) (0.2μ filtered following harvest in cell culture) is applied to a 5×10 cm column of Protein G at a linear flow rate of 199 cm/h. The column is previously washed with 100 mM glycine, pH 2.5 and equilibrated in 20 mM sodium phosphate, 150 mM sodium chloride, pH 7 prior to sample application. After the CM is loaded the column is washed with 5 column volumes of 20 mM sodium phosphate, 150 mM sodium chloride, pH 7 and eluted with 100 mM glycine, pH 2.5. The eluate is immediately neutralized with 3 M Tris, pH 8.5 and 0.2μ filtered.

Concentration/Dialysis—Protein G eluate is concentrated about 10 fold in an Amicon stirred cell fitted with a 30K membrane. The concentrate is dialyzed against buffer.

Factor Xa Cleavage and Purification to Generate Free Receptor—TR8-Fc is added to 50 μg of Factor Xa resulting in a 1:200 e:s ratio. The mixture is incubated overnight at 4° C.

Protein G Chromatography of the Free TR8 receptor—A 1 ml column of Protein G is equilibrated in 20 mM sodium phosphate, 150 mM sodium chloride, pH 6.5 in a disposable column using gravity flow. The cleaved receptor is passed over the column 3 times after which the column is washed with 20 mM sodium phosphate, 150 mM sodium chloride, pH 6.5 until no A₂₈₀ absorbence is seen. The column is eluted with 2.5 ml of 100 mM glycine, pH 2.5 neutralized with 83 μl of 3 M Tris, pH 8.5. TR8 elutes in the unbound fraction.

Concentration—The unbound fraction from the Protein G column is concentrated in a Centricon 10K cell (Amicon) to about a final concentration of 3.5 mg/ml estimated by A₂₈₀ extinction coefficient 0.7.

Mono S Chromatography—The concentrated sample is diluted to 5 ml with 20 mM sodium phosphate, pH 6 and applied to a 0.5×5 cm Mono S column equilibrated in 20 mM sodium phosphate, pH 6 at a linear flow rate of 300 cm/h. The column is washed with 20 mM sodium phosphate, pH 6 and eluted with a 20 column volume linear gradient of 20 mM sodium phosphate, pH 6 to 20 mM sodium phosphate, 1 M sodium chloride, pH 6. TR8 protein elutes in the unbound fraction.

Concentration/Dialysis—The unbound fraction from the Mono S column is concentrated to 1 ml as above using a Centricon 10K cell and is dialyzed against 20 mM sodium phosphate, 150 mM sodium chloride, pH 7.

Biotinylation—0.5 mg of TR8 at about 1-2 mg/ml is dialyzed against 100 mM borate, pH 8.5. A 20-fold molar excess of NHS-LC Biotin is added and the mixture is left on a rotator overnight at 4° C. The biotinylated TR8 is dialyzed against 20 mM sodium phosphate, 150 mM sodium chloride, pH 7, sterile filtered and stored at −70° C. Biotinylation is demonstrated on a Western blot probed with strepavidin HRP and subsequently developed with ECL reagent.

### Example 6

Characterization of the Intracellular Domain of TR8:Interaction with TRAFs and Activation of NF-κB and JNK

Various members of the TNF receptor superfamily interact directly with signaling molecules of the TNF receptor-associated factor (TRAF) family to elicit activation of NF-κB (nuclear factor κB) and the c-jun N-terminal kinase (JNK/SAPK) pathway. TR8, a TNF receptor family member and its ligand (TR8L) promotes survival of dendritic cells and differentiation of osteoclasts. TR8 contains 383 amino acids in its intracellular domain (amino acid residues 234-615; amino acid residues 209-590 of SEQ ID NO:2) in which resides three putative TRAF binding domains (termed I, II, and III). In this study, we examined the region of TR8 needed for interaction with TRAF molecules and for stimulation of NF-κB and JNK activity. We constructed epitope-tagged TR8 (F-TR8-615) and three C-terminal truncations (F-TR8-330, 427, and 530) lacking 85, 188, and 285 amino acids respectively. From this deletion analysis, TRAF2, TRAF5, and TRAF6 interact with TR8 at its C-terminal 85 amino acid tail, although the binding affinity appears to be in the order of TRAF2>>>TRAF5>TRAF6. Furthermore, overexpression of TR8 stimulates JNK and NF-κB activation. However, when the C-terminal tail which is necessary for TRAF binding was deleted, the truncated TR8 receptor was still capable of stimulating JNK activity, but not NF-κB, suggesting that interaction with TRAFs is necessary for NF-κB, but not necessarily for activation of the JNK pathway.

To date, over 20 members of the TNF ligand and receptor superfamilies have been identified. Most of these receptors activate signaling cascades including activation of NF-κB, protein kinases (MAPK/JNK/p38), and apoptosis through engagement of various adaptor proteins (Liu et al., Cell, 87:565-576 (1996); Damay et al., J. Leuk. Biol., 61:559-566 (1977); Song et al., Proc. Natl. Acad. Sci. 94:9792-9796 (1997)). Activation of apoptosis is typically transmitted through death domain containing receptors. Additionally, many of the TNFR family members activate NF-κB and JNK pathways via interaction with various TRAF family members (Liu et al., Cell 87:565-576 (1996); Song et al., Proc. Natl. Acad. Sci. 94:9792-9796 (1997); Cao et al., Nature 383:443-446 (1996); Hsu et al., J. Biol. Chem. 272:13471-13474 (1996); Ishida et al., J. Biol. Chem. 271:28745-28748 (1996); Marsters et al., J. Biol. Chem. 272:14029-14032 (1997); Rothe et al., Cell 78:681-692 (1994); Reinhard et al., EMBO J. 16:1080-1092 (1997); Natoli et al., Science 275:200-203 (1997); Rothe et al., Science 269:1424-1427 (1995)). The TRAF family consists of six distinct proteins which contain a Ring and zinc finger motif in their N-terminus and a C-terminal domain which appears to be responsible for self-association and protein interaction. TRAF family members TRAF1, TRAF2, and TRAF3 bind to distinct motifs within CD40, CD30, ATAR/HVEM, and p80 TNFR (Hsu et al., J. Biol. Chem. 272:13471-13474 (1996); Ishida et al., J. Biol. Chem. 271:28745-28748 (1996); Marsters et al., J. Biol. Chem. 272:14029-14032 (1997); Boucher et al., Biochem. Biophys. Res. Comm. 233:592-600 (1997)). The PXQXT/S motif is characteristic for binding TRAF1, TRAF2, and TRAF5 (Hsu et al., J. Biol. Chem. 272:13471-13474 (1996); Ishida et al., J. Biol. Chem. 271:28745-28748 (1996)). Moreover, TRAF6 interacts with CD40 via a 15 amino acid region (residues 230-245) (Ishida et al., J. Biol. Chem. 271:28745-28748 (1996)). Of these TRAF molecules, only TRAF2, TRAF5, and TRAF6 have been demonstrated to mediate signaling of NF-κB and JNK (Song et al., Proc. Natl. Acad. Sci. 94:9792-9796 (1997); Cao et al., Nature 383:443-446 (1996); Reinhard et al., EMBO J. 16:1080-1092 (1997); Natoli et al., Science 275:200-203 (1997)).

To further elucidate regions of the intracellular domain necessary for signaling by TR8, we constructed various C-terminal truncations of TR8 and transiently expressed them in human cultured cell lines to characterize their ability to activate JNK and NF-κB and for their ability to interact with various TRAF family members. From this deletion analysis, TRAF2, TRAF5, and TRAF6 interact with TR8 at its C-terminal 85 amino acid tail, although TRAF2 appears to bind preferentially. Furthermore, overexpression of TR8 stimulates JNK and NF-κB activation. However, when the C-terminal tail, which is necessary for TRAF binding, was deleted, the truncated TR8 receptor was still capable of stimulating JNK activity, but not NF-κB. These results suggest that TR8's interaction with TRAFs is necessary for NF-κB, but not for activation of the JNK pathway.

Experimental Procedures

Reagents, Cell lines, and Antibodies—HeLa, an epithelial carcinoma cell line, and 293, a human embryonic kidney cell line, were obtained from the American Type Culture Collection (Rockville, Md.) and cultured in MEM supplemented with 10% fetal bovine serum and antibiotics. Affinity-purified rabbit anti-TRAF2 (SC-876, C-20) and anti-JNK1 (SC-474, C-17) antibodies were obtained from Santa Cruz Biotechnology (Santa Cruz, Calif.). Goat anti-rabbit IgG-conjugated horseradish peroxidase was obtained from BioRad Laboratories (Hercules, Calif.). Anti-FLAG (monoclonal antibody M2) and anti-FLAG (M2) conjugated-agarose were obtained Eastman Kodak Co. (New Haven, Conn.). Goat anti-mouse IgG conjugated to horseradish peroxidase was obtained from Transduction Laboratories (Lexington, Ky.). Protein A/G sepharose was obtained from Pierce (Rockford, Ill.).

Expression Plasmids—The complete cDNA for TR8 (pSPORT3.0-TR8) was identified through a homology search of an expressed sequence tag (EST) cDNA database (Human Genome Sciences, Inc., Rockville, Md.) obtained from a primary dendritic cell cDNA library for proteins containing the cysteine-rich repeat characteristic of TNFR family members. To generate FLAG-tagged TR8-615, primers (5′-primer: CTAAGAAAGCTTTGTACCAGTGAGAAGCAT (SEQ ID NO: 13) and 3′-primer: GACGTAGTCGACTCAAGCCTTGGCCCCGCC (SEQ ID NO:14) were used in a PCR reaction with pSPORT3.0-TR8 to generate a PCR product that would encode residues 33-615 (lacking the signal sequence) and cloned into the HindIII/SalI site of the expression vector pCMVFLAG1 (Eastman Kodak Co., New Haven, Conn.). TR8 deletion mutants were generated by PCR using the above 5′ primer and the 3′ primers (TR8-330: TCCTACGTC GACTCAGCTGACCAATGAGAGAGCATCCT (SEQ ID NO:15); TR8-427: AACGGC GTCGACTCAACTGTCCACCTCTTTTTGCAA (SEQ ID NO:16); and TR8-530: CGCT GAGTCGACTCAGGAGTTACTTGTTTCCAGTCAC (SEQ ID NO: 17)) and cloned into the HindIII/SalI site of pCMVFLAG1. All plasmids were verified by automated DNA sequencing. The complete cDNA for TRAF2 was cloned by PCR using primers containing BamHI (5′) and SalI (3′) sites and pcDNA3HisTRAF2 as a template. The TRAF2 PCR product was digested with BamHI/SalI and cloned into pRKmyc resulting in pRKmycTRAF2. The cDNA for TRAF6 was digested from pSRα-TRAF6 with KpnI/EcoRI and cloned into pBS(KS-) to give rise to pBS-TRAF6.

In Vitro Translation of ³⁵S-Labeled TRAFs—Expression vectors encoding for TRAF2 (pRKmycTRAF2), TRAF5 (pcDNA3mycTRAF5), and TRAF6 (pBS-TRAF6) were in vitro transcribed and translated with ³⁵S-Met (Amersham, Chicago, Ill.) using the TNT system as described by the manufacturer (Promega, Madison, Wis.).

Transient Transfections—HeLa (1.5×10⁶ cells/100 mm dish) and 293 (2×10⁶ cells/100 mm dish) cells were plated the day before and transfected with 7.5-10 μg of expression vector by using Lipofectamine (GIBCO BRL, Gaithersburg, Md.) as described by the manufacturer and allowed to proceed for an additional 24 hrs. Alternatively, 293 cells (0.6×10⁶ cells/well, 6-well plate) were plated the day before and transfected the next day by calcium phosphate as described by the manufacturer (GIBCO BRL, Gaithersburg, Md.). Cells were harvested 36-40 hrs post-transfection and half of the cells were analyzed for expression of epitope-tagged receptors and JNK activities and the other half of the cells were analyzed for NF-κB by EMSAs. Lysates were prepared in lysis buffer (20 mM TRIS pH 8, 250 mM NaCl, 1 mM DTT, 2 mM EDTA, 1% Triton X-100, 10 g/ml leupeptin, 10 g/ml aprotinin, 0.5 mg/ml benzamidine, and 2 mM sodium vanadate). After 30 min. on ice, the samples were cleared by centrifugation for 10 min. Protein was estimated using a BioRad Protein determination kit (BioRad, Hercules, Calif.).

Western Blotting—Whole cell lysates (15 g) or proteins from immunoprecipitations were separated by 8.5% SDS-PAGE and electroblotted onto nitrocellulose membranes (BioRad, Hercules, Calif.). Western blot analysis was performed using the indicated antibodies, and membranes were developed by Enhanced Chemiluminescence (ECL) (Amersham, Chicago, Ill.).

Immunoprecipitations and JNK Kinase Assays—From transient transfected cells, lysates were prepared and immunoprecipitated using anti-FLAG-conjugated agarose or anti-JNK1 and protein A/G sepharose for 1 hr. Where indicated, ³⁵S-labeled proteins were added to the lysate prior to immunoprecipitation. Beads were collected by centrifugation and washed four times in lysis buffer followed by two washes in kinase buffer (20 mM TRIS, pH 8, 50 mM NaCl, and 1 mM DTT). For coimmunoprecipation, proteins were eluted in SDS-sample buffer, boiled, and subjected to SDS-PAGE. Analysis of JNK activity was performed using exogenously added GST-Jun(1-79) as a substrate as previously described (Haridas et al., J. Immunol. 160:3152-3162 (1998)). Quantitation of JNK activity and ³⁵S-labeled TRAF binding was analyzed using a PhosphoImager and Imagequant Software (Molecular Dynamics, Sunnyvale, Calif.).

Electrophoretic Mobility Shift Assays (EMSA)—Nuclear extracts were prepared from transfected cells essentially as described (Haridas et al., J. Immunol. 160:3152-3162 (1998)). Equivalent amounts of nuclear protein were used in a EMSA reaction with ³²P-labeled NF-κB oligonucleotide from the HIV-LTR as described (Haridas et al., J. Immunol., 160:3152-3162 (1998)). Quantitation of relative NF-κB activation was analyzed using a Phospholmager and Imagequant Software.

Results and Discussion

The full length TR8 encoding cDNA encodes a protein of 615 amino acid residues. The extracellular domain (residues 1-208 of FIGS. 1A-C; residues −25 to 183 of SEQ ID NO:2)) contains a signal sequence and the conserved cysteine rich repeats characteristic of the TNFR family (Vandenabeele et al., Trends Cell Biol. 5:392-399 (1995)). The intracellular domain (residues 234-615 of FIGS. 1A-C; residues 209-590 of SEQ ID NO:2) is the largest of all the TNFR family members to date and contains no homology to other members of this family.

Construction and Expression of Epitope-Tagged TR8—To facilitate detection and immunoprecipitation of TR8 in cultured cells, we constructed a FLAG epitope-tagged version of TR8 in the plasmid pCMVFLAG1. The mature polypeptide encoding residues 33-615 of FIGS. 1A-C; residues 8-590 of SEQ ID NO:2 (F-TR8-615) would be directed to the plasma membrane with a FLAG epitope tag at its N-terminus (FIG. 5A). To initially identify which region of the cytoplasmic domain is needed for signaling, we constructed three C-terminal deletions designated F-TR8-530, 427, and 330 (FIGS. 1A-C) lacking 85, 188, and 285 amino acids, respectively.

Most of the TNFR family members interact directly with various members of the TRAF family of signaling proteins. Of those receptors that bind to TRAF2, TRAF3, and TRAF5, a consensus TRAF binding motif (PxQxT/S) in the receptor is necessary for TRAF interaction (Ishida et al., J. Biol. Chem. 271:28745-28748 (1996); Boucher et al., Blochem. Biophys. Res. Comm. 233:592-600 (1997); Ishida et al., Proc. Natl. Acad. Sci. 93:9437-9442 (1996); Brodeur et al., J. Biol. Chem. 272:19777-19784 (1997)). By inspection of the intracellular domain of TR8, there appears to be three potential TRAF binding domains, two at the C-terminus (TRAFII and III) and one in the middle of the intracellular domain (TRAFIII) (FIG. 5B). Transient expression of F-TR8 and its deletion mutants was demonstrated in both HeLa, an epithelial carcinoma cell line, and 293, a human embryonic kidney cell line (data not shown). As expected, the deletion mutants were expressed similarily in both cell lines tested; however, expression levels of the deletion mutants was typically less than the full length receptor even using similar amounts of expression vectors.

TRAF2, TRAF5, and TRAF6 Interact with the C-Terminus of TR8—Since most of the TNFR family members utilize TRAFs as signaling components and that TR8 contains putative TRAF binding domains, we examined the ability for TR8 to interact with various TRAFs. We transiently transfected HeLa and 293 cells with vectors directing expression of F-TR8-615 and F-TR8 deletion mutants. After 24-36 hr, cell lysates were prepared and epitope-tagged receptors were immunoprecipitated with anti-FLAG conjugated-agarose. Coprecipitation of endogenous TRAF2 was detected by western blotting with anti-TRAF2 polyclonal antibodies. When expressed in HeLa and 293 cells, only F-TR8-615 routinely precipitated endogenous TRAF2, while none of the F-TR8 deletion mutants could precipitate endogenous TRAF2 (data not shown). Membranes were also probed with anti-FLAG to insure precipitation of epitope-tagged receptors (data not shown).

To examine whether other TRAFs could interact with TR8, we transiently transfected 293 cells with F-TR8 expression vectors. After 36 hr, cell lysates were prepared and in vitro translated ³⁵S-labeled TRAF2, TRAF5, and TRAF6 were added to each of the lysates. The epitope-tagged receptors were immunoprecipitated with anti-FLAG conjugated-agarose and bound proteins were eluted in SDS-sample buffer and subjected to SDS-PAGE. The bound ³⁵S-labeled TRAFs were detected by exposure of the dried SDS-PAGE gel to x-ray film. Like coprecipitation of endogenous TRAF2, only F-TR8-615 coprecipitated ³⁵S-labeled TRAF2 and to a lesser extent ³⁵S-labeled TRAF5 and TRAF6 (data not shown). Quantitation of ³⁵S-labeled TRAF2, TRAF5, and TRAF6 bound to F-TR8-615 resulted in a 145-, 11-, and 5-fold increase in binding relative to vector transfected cells, respectively. Thus, we have shown that TRAF2, TRAF5, and TRAF6 interact with TR8 at its C-terminal 85 residues.

TR8 Deletion Mutants Lacking TRAF Binding Domains (II and III) Activate JNK—TRAF2, TRAF5, and TRAF6 are involved in JNK activation (Song et al., Proc. Natl. Acad. Sci. 94:9792-9796 (1997)) by various members of the TNFR family and the interleukin-1 receptor (Cao et al., Nature 383:443-446 (1996)) (i.e., TRAF6). We tested whether other deletion mutants of TR8 lacking the C-terminus are capable of activation of JNK. Since several TNFR family members are capable of ligand-independent signaling when overexressed in cultured cell lines (Damay et al., J. Leuk. Biol. 61:559-566 (1997)), we transiently transfected 293 cells with increasing amounts of F-TR8 expression vectors. After 36 hr post-transfection, cell lysates were prepared and analyzed for receptor expression by western blotting with anti-FLAG antibodies (data not shown). Furthermore, the cell lysates were assayed for JNK activation by immune complex kinase assays using GST-Jun(1-79) as a substrate. Transient overexpression of F-TR8-615 in 293 cells leads to activation of JNK. Furthermore, F-TR8-530 and 427 deletion mutants lacking 85 and 188 residues from the C-terminus, respectively, could still activate JNK. However, C-terminal truncation of 285 residues (which leaves approximately 98 amino acids intact) could not activate JNK (data not shown). From at least three independent transfection experiments, we found that F-TR8-615, 530, and 427 could increase JNK activity between 4- to 10-fold, while F-TR8-330 was found not to exceed 1.5-fold relative to vector transfected samples. This data suggests that F-TR8-530 and F-TR8-427 may stimulate JNK activation in the absence of binding directly to TRAFs. Since F-TR8-330 could not stimulate JNK activation, we could tentatively localize a JNK activation domain between residues 330-427 of FIGS. 1A-C (residues 305-402 of SEQ ID NO:2) within the cytoplasmic domain of TR8.

TR8's C-Terminus is Necessary for NF-κB Activation—Overexpression of TR8 in 293 cells activates NF-κB as analyzed by gel mobility shift assays (Anderson et al., Nature 390:175-179 (1997)). To explore whether TR8 deletions mutants could activate NF-κB, we transiently transfected 293 cells with F-TR8-615 and the F-TR8 deletions mutants. Cells were harvested 36 hr post-transfection and half of the cells were used for JNK and western blotting, while the other half of the cells were used to prepare nuclear extracts. Western blotting with anti-FLAG antibodies indicated expression of the epitope-tagged receptors and JNK immune complex kinase assays indicated stimulation of JNK activity. Analysis of NF-κB by a gel mobility shift assay indicated that only F-TR8-615 could activate NF-κB (data not shown). None of the F-TR8 deletions were capable of activating NF-κB in three independent transient transfection experiments, even though from the same transfections F-TR8-530 and 427 could activate JNK.

Our data is consistent with a previous report (Anderson et al., Nature 390:175-179 (1997), indicating that transient overexpression of TR8 in 293 cells induces NF-κB. We further demonstrated deletion of the C-terminal 85 residues, which is necessary for TRAF interaction, appears also to be necessary for NF-κB activation. Whether the interaction between TR8 and TRAFs is responsible for NF-κB activation remains to be determined. Our data is in agreement with reports which show that TRAF2, TRAF5, and TRAF6 participate in NF-κB activation by other TNFR family members (Song et al., Proc. Natl. Acad. Sci. 94:9792-9796 (1997)).

Stimulation of mouse thymocytes or T-cells, but not B-cells, by TR8L/TRANCE induces JNK activation (Wong et al., J. Biol. Chem. 272:25190-25194 (1997)) which could be inhibited in thymocytes from transgenic mice expressing a dominant negative form of TRAF2 (Wong et al., J. Exp. Med. 186:2075-2080 (1997)). From our deletion analysis of TR8, we provided evidence that TR8 lacking the TRAF binding domain could still stimulate JNK activity. Furthermore, our deletion analysis implicates residues between 330-427 of FIGS. 1A-C (305-402 of SEQ ID NO:2) of TR8 to be necessary for JNK activation. Thus, it appears that TR8 can activate JNK in a TRAF-independent manner. This may be contradictory to that published by ligand stimulation of thymocytes from dominant negative TRAF2 transgenic mice (Wong et al., J. Exp. Med. 186:2075-2080 (1997)), however, the experimental conditions are too different to compare these two sets of results. It is possible that TR8 can activate the JNK pathway in both a TRAF-dependent and -independent fashion. Moreover, it is also possible that other unidentified adaptor proteins and TRAF-like molecules are responsible for signaling by TR8.

In summary, TR8 encodes the largest cytoplasmic domain (383 amino acids) of any TNFR family member thus far. For the first time, we provide evidence that TRAF2, TRAF5, and TRAF6 bind to the C-terminal 85 amino acids, however TRAF2 appears to bind better than TRAF5 and TRAF6. Furthermore, we demonstrated that deletion of the TRAF interaction motif at the C-terminus, did not inhibit TR8 from stimulation of JNK activity, suggesting that TR8 could potentially activate JNK in a TRAF-independent manner. However, deletion of the C-terminal 85 residues results in loss of NF-κB activation. Thus, we have demonstrated that TRAF family members interact with the novel TNFR family member TR8, and could possibly participate in TR8 signal transduction.

It will be clear that the invention may be practiced otherwise than as particularly described in the foregoing description and examples.

Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. An isolated nucleic acid molecule comprising a polynucleotide having a nucleotide sequence at least 95% identical to a sequence selected from the group consisting of:
(a) a nucleotide sequence encoding the TR8 receptor polypeptide having the amino acid sequence at positions from about −25 to about 590 in SEQ ID NO:2;
(b) a nucleotide sequence encoding the TR8 receptor polypeptide having the amino acid sequence at positions from about −25 to about 211 in SEQ ID NO:2;
(c) a nucleotide sequence encoding the TR8 receptor polypeptide having the amino acid sequence at positions from about −5, −3 or +1 to about 590 in SEQ ID NO:2;
(d) a nucleotide sequence encoding the TR8 receptor polypeptide having the amino acid sequence encoded by the cDNA clone contained in ATCC Deposit Number 97956;
(e) a nucleotide sequence encoding the mature TR8 receptor having the amino acid sequence encoded by the cDNA clone contained in ATCC Deposit Number 97956;
(f) a nucleotide sequence encoding the TR8 extracellular domain;
(g) a nucleotide sequence encoding the TR8 transmembrane domain;
(h) a nucleotide sequence encoding the TR8 intracellular domain; and
(I) a nucleotide sequence complementary to any of the nucleotide sequences in (a), (b), (c), (d), (e), (f), (g) or (h) above.

2. The nucleic acid molecule of claim 1 wherein said polynucleotide has the nucleotide sequence in SEQ ID NO: 1.

3. The nucleic acid molecule of claim 1 wherein said polynucleotide has the nucleotide sequence in SEQ ID NO: 1 encoding a polypeptide having the complete amino acid sequence in SEQ ID NO:2.

4. The nucleic acid molecule of claim 1 wherein said polynucleotide has the nucleotide sequence in SEQ ID NO: 1 encoding the mature TR8 receptor having the mature amino acid sequence in SEQ ID NO:2.

5. The nucleic acid molecule of claim 1 wherein said polynucleotide has the complete nucleotide sequence of the cDNA clone contained in ATCC Deposit Number 97956.

6. The nucleic acid molecule of claim 1 wherein said polynucleotide has the nucleotide sequence encoding a TR8 receptor polypeptide having the amino acid sequence encoded by the cDNA clone contained in ATCC Deposit Number 97956.

7. The nucleic acid molecule of claim 1 wherein said polynucleotide has the nucleotide sequence encoding the mature TR8 receptor polypeptide having the amino acid sequence encoded by the cDNA clone contained in ATCC Deposit Number 97956.

8. An isolated nucleic acid molecule comprising a polynucleotide which hybridizes under stringent hybridization conditions to a polynucleotide having a nucleotide sequence identical to a nucleotide sequence in (a), (b), (c), (d), (e), (f), (g) or (h) of claim 1 wherein said polynucleotide which hybridizes does not hybridize under stringent hybridization conditions to a polynucleotide having a nucleotide sequence consisting of only A residues or of only T residues.

9. An isolated nucleic acid molecule comprising a polynucleotide which encodes the amino acid sequence of an epitope-bearing portion of a TR8 receptor having an amino acid sequence in (a), (b), (c), (d), (e), (f), (g) or (h) of claim 1.

10. The isolated nucleic acid molecule of claim 9, which encodes an epitope-bearing portion of a TR8 receptor polypeptide selected from the group consisting of: a polypeptide comprising amino acid residues from about 10 to about 65 in SEQ ID NO:2; a polypeptide comprising amino acid residues from about 82 to about 185 in SEQ ID NO:2; a polypeptide comprising amino acid residues from about 211 to about 257 in SEQ ID NO:2; a polypeptide comprising amino acid residues from about 267 to about 512 in SEQ ID NO:2; and a polypeptide comprising amino acid residues from about 531 to about 590 in SEQ ID NO:2.

11. The isolated nucleic acid molecule of claim 1, which encodes a TR8 receptor extracellular domain.

12. The isolated nucleic acid molecule of claim 1, which encodes a TR8 receptor transmembrane domain.

13. The isolated nucleic acid molecule of claim 1, which encodes a TR8 receptor intracellular domain.

14. A method for making a recombinant vector comprising inserting an isolated nucleic acid molecule of claim 1 into a vector.

15. A recombinant vector produced by the method of claim 14.

16. A method of making a recombinant host cell comprising introducing the isolated nucleic acid molecule of claim 1 into a host cell.

17. A recombinant host cell produced by the method of claim 16.

18. A recombinant method for producing a TR8 polypeptide, comprising culturing the recombinant host cell of claim 17 under conditions such that said polypeptide is expressed and recovering said polypeptide.

19. An isolated nucleic acid molecule comprising a polynucleotide having a nucleotide sequence in SEQ ID NO: 1 wherein an additional CGC codon is inserted after nucleotide 72 resulting in insertion of an additional R residue after position 3 in SEQ ID NO:2; nucleotide 763 is G instead of A, resulting in the amino acid E instead of F at position 194 in SEQ ID NO:2; and nucleotide 1583 is G instead of T, resulting in the amino acid S instead of I at position 487 in SEQ ID NO:2.

20. An isolated TR8 receptor polypeptide having the amino acid sequence in SEQ ID NO:2 wherein an additional R residue is inserted after position 3 in SEQ ID NO:2; the amino acid at position 194 in SEQ ID NO:2 is E instead of F; and the amino acid at position 487 in SEQ ID NO:2 is S instead of I.

21. An isolated TR8 polypeptide having an amino acid sequence at least 95% identical to a sequence selected from the group consisting of:
(a) the amino acid sequence of the TR8 polypeptide having the amino acid sequence at positions from about −25 to about 590 in SEQ ID NO:2;
(b) the amino acid sequence of the TR8 polypeptide having the amino acid sequence at positions from about −25 to about 211 in SEQ ID NO:2;
(c) the amino acid sequence of the TR8 polypeptide having the amino acid sequence at positions from about −5, −3 or +1 to about 590 in SEQ ID NO:2;
d) the amino acid sequence of the TR8 polypeptide having the complete amino acid sequence encoded by the cDNA clone contained in ATCC Deposit Number 97956;
(e) the amino acid sequence of the mature TR8 polypeptide having the amino acid sequence encoded by the cDNA clone contained in ATCC Deposit Number 97956;
(f) the amino acid sequence of the TR8 receptor extracellular domain;
(g) the amino acid sequence of the TR8 receptor transmembrane domain;
(h) the amino acid sequence of the TR8 receptor intracellular domain; and
(q) the amino acid sequence of an epitope-bearing portion of any one of the polypeptides of (a), (b), (c), (d), (e), (f), (g) or (h).

22. An isolated polypeptide comprising an epitope-bearing portion of a TR8 receptor protein, wherein said portion is selected from the group consisting of: a polypeptide comprising amino acid residues from about 10 to about 65 in SEQ ID NO:2; a polypeptide comprising amino acid residues from about 82 to about 185 in SEQ ID NO:2; a polypeptide comprising amino acid residues from about 211 to about 257 in SEQ ID NO:2; a polypeptide comprising amino acid residues from about 267 to about 512 in SEQ ID NO:2; and a polypeptide comprising amino acid residues from about 531 to about 590 in SEQ ID NO:2.

23. An isolated antibody that binds specifically to a TR8 receptor polypeptide of claim 21.

24. A method of treating herpes simplex viral infection comprising introducing an effective amount of a soluble fragment of a TR8 polypeptide into an individual to be treated in admixture with a pharmaceutically acceptable carrier.

25. A method of treating a disease state associated with aberrant cell survival comprising introducing an effective amount of a TR8 protein, or agonist or antagonist thereof, into an individual to be treated in admixture with a pharmaceutically acceptable carrier.

26. A method of screening for agonists and antagonists of TR8 activity comprising:
(a) contacting cells which express a TR8 receptor with a candidate compound,
(b) assaying a cellular response, and
(c) comparing the cellular response to a standard cellular response made in absence of the candidate compound; whereby, an increased cellular response over the standard indicates that the compound is an agonist and a decreased cellular response over the standard indicates that the compound is an antagonist.


SEQUENCE LISTING (24 sequences)

SEQ ID NO: 1 - DNA, 2853 bp, human
gtcgacccacgcgtccgggccgcggcgcccgccagcctgtcccgcgccatggccccgcgcgcccggcgccgcccgctgttcgcgctgctgctgctctgcgcgctgctcgcccggctgcaggtggctttgcagatcgctcctccatgtaccagtgagaagcattatgagcatctgggacggtgctgtaacaaatgtgaaccaggaaagtacatgtcttctaaatgcactactacctctgacagtgtatgtctgccctgtggcccggatgaatacttggatagctggaatgaagaagataaatgcttgctgcataaagtttgtgatacaggcaaggccctggtggccgtggtcgccggcaacagcacgaccccccggcgctgcgcgtgcacggctgggtaccactggagccaggactgcgagtgctgccgccgcaacaccgagtgcgcgccgggcctgggcgcccagcacccgttgcagctcaacaaggacacagtgtgcaaaccttgccttgcaggctacttctctgatgccttttcctccacggacaaatgcagaccctggaccaactgtaccttccttggaaagagagtagaacatcatgggacagagaaatccgatgtggtttgcagttcttctctgccagctagaaaaccaccaaatgaaccccatgtttacttgcccggtttaataattctgcttctcttcgcgtctgtggccctggtggctgccatcatctttggcgtttgctataggaaaaaagggaaagcactcacagctaatttgtggcactggatcaatgaggcttgtggccgcctaagtggagataaggagtcctcaggtgacagttgtgtcagtacacacacggcaaactttggtcagcagggagcatgtgaaggtgtcttactgctgactctggaggagaagacatttccagaagatatgtgctacccagatcaaggtggtgtctgtcagggcacgtgtgtaggaggtggtccctacgcacaaggcgaagatgccaggatgctctcattggtcagcaagaccgagatagaggaagacagcttcagacagatgcccacagaagatgaatacatggacaggccctcccagcccacagaccagttactgttcctcactgagcctggaagcaaatccacacctcctttctctgaacccctggaggtgggggagaatgacagtttaagccagtgcttcacggggacacagagcacagtgggttcagaaagctgcaactgcactgagcccctgtgcaggactgattggactcccatgtcctctgaaaactacttgcaaaaagaggtggacagtggccattgcccgcactgggcagccagccccagccccaactgggcagatgtctgcacaggctgccggaaccctcctggggaggactgtgaacccctcgtgggttccccaaaacgtggacccttgccccagtgcgcctatggcatgggccttccccctgaagaagaagccagcaggacggaggccagagaccagcccgaggatggggctgatgggaggctcccaagctcagcgagggcaggtgccgggtctggaatctcccctggtggccagtcccctgcatctggaaatgtgactggaaacagtaactccacgttcatctccagcgggcaggtgatgaacttcaagggcgacatcatcgtggtctacgtcagccagacctcgcaggagggcgcggcggcggctgcggagcccatgggccgcccggtgcaggaggagaccctggcgcgccgagactccttcgcggggaacggcccgcgcttcccggacccgtgcggcggccccgaggggctgcgggagccggagaaggcctcgaggccggtgcaggagcaaggcggggccaaggcttgagcgccccccatggctgggagcccgaagctcggagccagggctcgcgagggcagcaccgcagcctctgccccagccccggccacccagggatcgatcggtacagtcgaggaagaccacccggcattctctgcccactttgccttccaggaaatgggcttttcaggaagtgaattgatgaggactgtccccatgcccacggatgctcagcagcccgccgcactggggcagatgtctcccctgccactcctcaaactcgcagcagtaatttgtggcactatgacagctatttttatgactatcctgttctgtgggggggggggtctgttttccccccatatttgtattccttttcataacttttcttgatatctttcctccctcttttttaatgtaaaggttttctcaaaaattctcctaaaggtgagggtctctttcttttctcttttccttttttttttctttttttggcaacctggctctggcccaggctagagtgcagtggtgcgattatagcccggtgcagcctctaactcctgggctcaagcaatccaagtgatcctcccacctcaaccttcggagtagctgggatcacagctgcaggccacgcccagcttcctccccccgactccccccccagagacacggtcccaccatgttaacccagcctggtctcaaactcacccagtaaagcagtcctaccagcctcggcctcccaaagtcactgggattcacaggcgtgagcccccacgctggcctgctttacgtattttcttttgtgcccctgctcacagtgttttagagatggctttcccagtgtgtgttcattgtaaacacttttgggaaagggctaaacatgtgaggcctggagatagttgctaagttgctaggaacatgtggtgggactttcatattctgaaaaatgttctatattctcatttttctaaaaaaaaaaaaaaa

SEQ ID NO: 2 - protein, 615 aa, human
MAPRARRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDVVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGISPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 3 - protein, 450 aa, human
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASRDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLP

SEQ ID NO: 4 - protein, 277 aa, human
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 5 - protein, 277 aa, human
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 6 - protein, 435 aa, human
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 7 - DNA, 27 bp, human
cgcccatggctttgcagatcgctcctc

SEQ ID NO: 8 - DNA, 27 bp, human
cgcaagcttttagggcaagtaaacatg

SEQ ID NO: 9 - DNA, 34 bp, human
cgcggatccgccatcatggccccgcgcgcccggc

SEQ ID NO: 10 - DNA, 27 bp, human
cgcggtaccttagggcaagtaaacatg

SEQ ID NO: 11 - DNA, 27 bp, human
cgcggtaccctgcgagtttgaggagtg

SEQ ID NO: 12 - DNA, 57 bp, human
cgctctagatcaagcgtagtctgggacgtcgtatgggtattagggcaagtaaacatg

SEQ ID NO: 13 - DNA, 30 bp, human
ctaagaaagctttgtaccagtgagaagcat

SEQ ID NO: 14 - DNA, 30 bp, human
gacgtagtcgactcaagccttggccccgcc

SEQ ID NO: 15 - DNA, 38 bp, human
tcctacgtcgactcagctgaccaatgagagagcatcct

SEQ ID NO: 16 - DNA, 36 bp, human
aacggcgtcgactcaactgtccacctctttttgcaa

SEQ ID NO: 17 - DNA, 37 bp, human
cgctgagtcgactcaggagttacttgtttccagtcac

SEQ ID NO: 18 - protein, 15 aa, human
HTPHYPEQETEPPLG

SEQ ID NO: 19 - protein, 15 aa, human
SNTAAPVQETLHGCQ

SEQ ID NO: 20 - protein, 15 aa, human
DSLPHPQQATDSGHE

SEQ ID NO: 21 - protein, 15 aa, human
DVTTVAVEETIPSFT

SEQ ID NO: 22 - protein, 15 aa, human
EYMDRPSQPTDQLLF

SEQ ID NO: 23 - protein, 15 aa, human
EPMGRPVQEETLARR

SEQ ID NO: 24 - protein, 15 aa, human
EKASRPVQEQGGAKA